SEQUENCE LISTING (18 sequences)

SEQ ID NO: 1 - protein, 116 aa, Artificial
  Inventor Ueda, Hiroshi; Abe, Ryoji Inventor Ihara, Masaki; Takagi, Hiroaki
QVKLQQSGAEFVKAGASVKLSCKTSGYTFNNYWIHWVKQSPGQGLEWIGEIDPSDGYSNYNQKFKGKATLTVDKSSSTAYMHLNSLTSEDSAVYYCTSSTSVGGSWGQGTTVTVSS

SEQ ID NO: 2 - protein, 113 aa, Artificial
  anti-BGP antibody VL
DIELTQSPLSLPVSLGDQASISCTSSQSLLHSNGDTYLHWYLQKPGQSPKLLIYTLSNRFSGVPDRFSGSGSGTDFTLKISRVEAADLGIYFCSQTTHVPYTFGGGTKLEIKR

SEQ ID NO: 3 - protein, 13 aa, Artificial
  ProXtag
MSKQIEVNXSNET

SEQ ID NO: 4 - protein, 8 aa, Artificial
  Spacer
GGGSGGGS

SEQ ID NO: 5 - protein, 21 aa, Artificial
  Linker
LVTVSSGGGGSGGGGSGGGGS

SEQ ID NO: 6 - protein, 119 aa, Artificial
  anti-BPA antibody VH
DVQLQESGPGLVKPSQSLSLTCSVTGYSITSGYYWNWIRQFPGNKLEWMGYIRYDGSNNYNPSLKNRISITRDTSKNQFFLKLNSVTPEDTATYYCARVLGRGYGLDYWGQGTSVTVSS

SEQ ID NO: 7 - protein, 111 aa, Artificial
  anti-BPA antibody VL
DIVLTQSPASLAVSLGQRATISCRASQSVSTSTYSYLHWYQQRPGQPPKLIKYVSNLESGVPARFSGSGSGTDFTLNIHPVEEEDTATYYCQHSWEIPPTFGGGTKLEIKR

SEQ ID NO: 8 - protein, 7 aa, Artificial
  BGP C-terminal peptide
RRFYGPV

SEQ ID NO: 9 - protein, 15 aa, Artificial
  linker
GGGGSGGGGSGGGGS

SEQ ID NO: 10 - protein, 12 aa, Artificial
  linker
GGGSGGGSGGGS

SEQ ID NO: 11 - protein, 20 aa, Artificial
  linker
GGGSGGGSGGGSGGGSGGGS

SEQ ID NO: 12 - protein, 113 aa, Artificial
  anti-HEL antibody VH
EVQLQESGPSLVKPSQTLSLTCSVTGDSITSDYWSWIRKFPGNRLEYMGYVSYSGSTYYNPSLKSRISITRDTSKNQYYLDLNSVTTEDTATYYCANWDGDYWGQGTLVTVSS

SEQ ID NO: 13 - protein, 108 aa, Artificial
  anti-HEL antibody-VL
DIVLTQSPATLSVTPGNSVSLSCRASQSIGNNLHWYQQKSHESPRLLIKYASQSISGIPSRFSGSGSGTDFTLSINSVETEDFGMYFCQQSNSWPYTFGGGTKLEIKR

SEQ ID NO: 14 - protein, 116 aa, Artificial
  anti-Estradiol antibody-VH
EVQLQQSGPELVKPGASVKISCKTSGYTFTKYTIHWVKQTHGKSLEWIGGINPINGGTSYNQKFKDKASLTVDKSSSTAYMELRSLTSKDSAVYYCEIYYGSFDHWGQGTTLTVSS

SEQ ID NO: 15 - protein, 109 aa, Artificial
  anti-Estradiol antibody-VL
DIVLTQSPAIMSAAPGEKVTLTCSASSSVSSSKLFWYQQKPGSSPKLWIYSTSNLASGVPARFSGSGSGTSYSLTISNMEAEDAASYFCLQWSGYPPTFGAGTKLEIKR

SEQ ID NO: 16 - protein, 116 aa, Artificial
  anti-BSA antibody-VH
EVQLLESGGGLVQPGGSLRLSCAASGFTFSSYAMSWVRQAPGKGLEWVSHISPYGANTRYADSVKGRFTISRDNSKNTLYLQMNSLRAEDTAVYYCAKGLRAFDYWGQGTLVTVSS

SEQ ID NO: 17 - protein, 109 aa, Artificial
  anti-BSA antibody-VL
TDIQMTQSPSSLSASVGDRVTITCRASQSISSYLNWYQQKPGKAPKLLIYRASLLQSGVPSRFSGSGSGTDFTLTISSLQPEDFATYYCQQGRDRPATFGQGTKVEIKR

SEQ ID NO: 18 - protein, 4 aa, Artificial
  Spacer
GGGS

(19) United States
(12) Patent Application Publication
(10) Pub. No.: US 2016/0349266 A1
(43) Pub. Date: Dec. 1, 2016

(54) Fluoroimmunoassay Method

(71) Applicant: USHIO DENKI KABUSHIKI KAISHA, Tokyo-to (JP)

(72) Inventors: Hiroshi Ueda, Tokyo (JP); Ryoji Abe, Chiba (JP); Masaki Ihara, Nagano (JP); Hiroaki Takagi, Chiba (JP)

(73) Assignee: USHIO DENKI KABUSHIKI KAISHA, Tokyo-to (JP)

(21) Appl. No.: 15/183,974

(22) Filed: Jun. 16, 2016

Related U.S. Application Data
(62) Division of application No. 13/510,105, filed on Jul. 5, 2012.

(30) Foreign Application Priority Data
Nov. 19, 2009 (JP) 2009-264420

(51) Int. Cl. G01N 33/58; G01N 21/64; C07K 16/18; C07K 16/44; G01N 33/68; A61K 49/00

(52) U.S. Cl. CPC G01N 33/582; G01N 33/6857; A61K 49/0058; C07K 16/18; C07K 16/44; G01N 21/6428; C07K 2317/56; C07K 2317/622; C07K 2317/92

(57) ABSTRACT

An object is to provide an immunoassay method requiring neither a solid-phase immobilization step nor a washing step, enabling quick and simple quantitative measurement of a target substance in a liquid phase and capable of visualizing an antigen. Such an object is attained by measuring the concentration of a target antigen present in a test substance by sequentially performing a step (a) of bringing an antibody light-chain variable region polypeptide and an antibody heavy-chain variable region polypeptide labeled with a fluorescent dye into contact with an antigen in a test substance in a liquid phase; or bringing an antibody heavy-chain variable region polypeptide and an antibody light-chain variable region polypeptide labeled with a fluorescent dye into contact with an antigen in a test substance in a liquid phase; a step (b) of measuring the fluorescence intensity of the fluorescent dye; and a step (c) of computationally obtaining the level of the antigen contained in the test substance with reference to a positive correlation between the concentration of the antigen in a liquid phase and the fluorescence intensity of the fluorescent dye.

### TECHNICAL FIELD

The present invention relates to a novel antigen-concentration measuring method requiring neither a solid-phase immobilization step nor a washing step, and a kit and the like for carrying out the antigen-concentration measuring method.

### BACKGROUND ART

Of methods for measuring concentrations of antigens and antibodies, a most widely used measuring method in clinical diagnosis, fundamental studies and environmental researches is an immunoassay method called a sandwich ELISA method (or sandwich RIA method), which uses two types of monoclonal antibodies recognizing different epitopes of a same antigen or uses a monoclonal antibody and a polyclonal antibody. The sandwich method will be more specifically described as follows. As a first stage, a mono/polyclonal antibody called as a primary antibody is immobilized to a measurement plate. To the plate, a test sample containing an antigen is supplied and allowed to react for a predetermined time to bind the antibody and the antigen. Then, as a second stage, contaminants bound to the antibody and antigen non-specifically bound to the plate are washed away with a washing liquid. As a third stage, a solution of a secondary antibody previously labeled with a reporter molecule such as an enzyme, a fluorescent dye or a radioisotope is supplied and allowed to react for a predetermined time. In this manner, to the antigen captured by the primary antibody, a labeled secondary antibody is further bound. After completion of the reaction, excessive labeled antibody is washed away with a washing liquid and the amount of reporter molecule bound to the measurement plate is measured based on e.g., enzyme activity, fluorescence or radioisotope. In this manner, the level of an antigen in the test sample is measured.

As described above, in a general sandwich ELISA method, two types of antibodies recognizing different epitopes are required. For example, in the case where e.g., a low-molecular compound, is used as an antigen, it is difficult to prepare a plurality of antibodies recognizing different epitopes. Therefore, Ueda et al. established a highly accurate immunoassay method for a low-molecular compound called an open-sandwich method, using a heavy-chain variable region (VH) and a light-chain variable region (VL) of a single antibody (Patent Documents 1 and 2, Non-patent Documents 1 and 2). This method is an antigen-concentration measuring method, which includes preparing a VH-region polypeptide and a VL-region polypeptide of an antibody specifically recognizing an antigen, labeling one of the polypeptides with a reporter molecule to prepare a labeled polypeptide, immobilizing the other polypeptide to a solid phase to prepare an immobilized polypeptide, bringing an antigen-containing sample and the labeled polypeptide into contact with the immobilized polypeptide, and measuring the amount of reporter molecule of the labeled polypeptide bound to the immobilized polypeptide. Furthermore, as a method for measuring a low-molecular compound, e.g., liquid chromatography is known other than the immunoassay method. However, the liquid chromatography requires a highly accurate measurement apparatus and a large amount of test sample and takes a long time for measurement. In addition, its versatility is low.

Furthermore, as an immunoassay method for measuring the concentration of an antigen using a fluorescent-dye labeled antibody, the following methods are known: an immunoassay method (Non-patent Documents 3 and 4), which includes labeling an antibody and an antigen with different fluorescent dyes, respectively, and using a change in efficiency of fluorescent resonance energy transfer (FRET) caused between the fluorescent dyes as a reference; an immunoassay method, which uses a phenomenon where fluorescence of a fluorescence-labeled antibody previously quenched by adding a quencher to the antibody is increased by introduction of a target detection substance, in short, using a change in efficiency by quenching as a reference; and a fluoroimmunoassay method (Patent Document 3), which measures a decrease of fluorescence intensity caused by aggregation of a measuring target substance with an antibody labeled with a fluorescent dye.

### PRIOR ART DOCUMENTS

### Patent Documents

Patent Document 1: Japanese Unexamined Patent Application Publication No. 10-78436
Patent Document 2: Japanese Patent No. 3784111
Patent Document 3: Japanese Unexamined Patent Application Publication No. 10-282098

### Non-Patent Documents

Non-patent Document 1: Hiroshi Ueda, Journal of the Pharmaceutical Society of Japan 27: 71-80 (2007)
Non-patent Document 2: Lim S L, et al., Anal Chem. 79 (16): 6193-200 (2007)
Non-patent Document 3: Iijima I. and Hohsaka T., Chembiochem. 17; 10 (6): 999-1006 (2009)
Non-patent Document 4: Kajihara D, et al., Nat Methods. 3 (11): 923 (2006)

### SUMMARY OF THE INVENTION

### Object to be Solved by the Invention

Immunoassay methods known up to present, as described above, all require a step of immobilizing an antibody or an antigen to a solid phase and a washing step of removing a labeled compound non-specifically adsorbed. These steps require intricate work and consume time. In addition, the results vary due to these steps. For these reasons, it has been desired to develop a liquid-phase immunoassay method requiring neither a solid-phase immobilization step nor a washing step. An object of the present invention is to provide an immunoassay method requiring neither a solid-phase immobilization step nor a washing step, enabling quick and simple quantitative measurement of a target substance in a liquid phase and capable of visualizing an antigen.

### Means to Solve the Object

The present inventors first attempted to establish an antibody/antigen binding activity evaluation system using antibody VH and VL labeled with different fluorescent dyes with reference to fluorescent resonance energy transfer (FRET) efficiency. In the FRET measurement, a fluorescent-dye CR110-labeled anti-BGP antibody light-chain region (CR110-VL) and a fluorescent-dye TAMRA-labeled anti-BGP antibody heavy-chain region (TAMRA-VH) were used. The inventors predicted that if an antigen is not present, FRET from CR110 to TAMRA will not occur; whereas, if an antigen is present, VH and VL form a ternary complex with the antigen between them, with the result that FRET from CR110 to TAMRA will occur. Based on such a prediction, the inventors incubated CR110-VL and TAMRA-VH or non-labeled VH, together with BGP antigen peptides in different concentrations and analyzed a change of fluorescence intensity of CR110 in accordance with fluorescence intensity multiple distribution analysis (FIDA). As a result, when CR110-VL was reacted with TAMRA-VH, the fluorescence intensity of CR110 decreased in a BGP antigen peptide concentration-dependent manner. From this, it was confirmed that CR110-VL and TAMRA-VH were bound with the antigen peptide between them to form a complex and FRET from CR110 to TAMRA occurred as predicted.

In contrast, unexpectedly, when CR110-VL was reacted with non-labeled VH, the fluorescence intensity of CR110 increased in a BGP antigen peptide concentration-dependent manner. The present inventors, for the purpose of determining whether the same phenomenon is observed when labeled VH is used, incubated TAMRA-VH and CR110-VL or non-labeled VL together with BGP antigen peptides in different concentrations and analyzed a change in fluorescence intensity of TAMRA. As a result, it was found that also in the case where TAMRA-VH was reacted with non-labeled VL, the fluorescence intensity of TAMRA-VH increased in a BGP antigen peptide concentration-dependent manner. These results were totally beyond expectation. The present inventors postulated that VL and VH may serve as a quencher for a fluorescent dye (CR110, TAMRA) and quench is eliminated only when VH and VL form a complex with an antigen peptide between them, with the result that the fluorescence intensity may increase.

Based on the hypothesis, in attempt to establish a novel assay method (hereinafter sometimes referred also to as “homogenous fluorescence based immunoassay”) using the quenching phenomenon, the present inventors reacted TAMRA-VH and BGP peptides in different concentrations in the presence or absence of non-labeled VL and measured the fluorescence intensity. As a result, the fluorescence intensity of TAMRA-VH increased in a BGP peptide concentration-dependent manner in the presence of VL. They analyzed the ratio of fluorescence intensity (+VL/−VL) of TAMRA-VH in the presence of VL to TAMRA-VH in the absence of VL. Based on the results, they found that a highly sensitive homogenous fluorescence based immunoassay could be constructed. Furthermore, the inventors determined that the sensitivity of the “homogenous fluorescence based immunoassay” increased by using ATTO655 as a fluorescent dye and labeling the fluorescent dye to VH with a spacer interposed between them.

Furthermore, the present inventors conducted an experiment for determining a quenching effect by use of mutant VH in which four tryptophan residues (hereinafter sometimes referred to as Trp or W) present in VH are each substituted with phenylalanine residue (hereinafter sometimes referred to as Phe or F) and demonstrated that tryptophan residues at the positions 36, 47, 106 (corresponding to the position 103 in the Kabat numbering system) in an amino acid sequence of VH serve as a quencher for a fluorescent dye. Since these tryptophan residues are highly conserved in mouse antibody VH, it was found that an antigen could be measured by use of various antibodies if the “homogenous fluorescence based immunoassay” of the present invention is applied.

The present invention has been attained based on the aforementioned findings.

### Effect of the Invention

According to the present invention, it is possible to provide an immunoassay method enabling quick and simple quantitative measurement of a target substance in a liquid phase and a kit for measuring an antigen by the assay method. The assay method of the present invention is a method of detecting/measuring binding of an antigen, antibody VL and VH with reference to the fluorescence intensity of a fluorescent dye labeled to the antibody VL or VH and based on the novel finding that when the antibody VL and VH are not bound, the fluorescent dye is in a quenched state; whereas, when the antibody VL and VH are bound with an antigen interposed between them, the quenched state of the fluorescent dye is eliminated. This method requires neither a solid-phase immobilization step nor a washing step indispensable in conventional immunoassay methods. Thus, highly accurate measurement results with less variability can be obtained in a short time.

More specifically, the present invention relates to (1) an antigen-concentration measurement/detection kit having an antibody light-chain variable region polypeptide and an antibody heavy-chain variable region polypeptide, either one of which is labeled with a fluorescent dye, wherein the kit enables a measurement of antigen concentration or visualization of an antigen with reference to the positive correlation between the concentration of the antigen in a liquid phase and the fluorescence intensity of the fluorescent dye; and (2) the antigen-concentration measurement/detection kit according to (1) above, in which the antibody heavy-chain variable region polypeptide and the antibody light-chain variable region polypeptide are linked to form a single-chain antibody.

Furthermore, the present invention relates to (3) the antigen-concentration measurement/detection kit according to (1) or (2) above, in which the fluorescent dye is a rhodamine-based fluorescent dye or an oxazine-based fluorescent dye; (4) the antigen-concentration measurement/detection kit according to (3) above, in which the fluorescent dye is CR110, TAMRA or ATTO655; (5) the antigen-concentration measurement/detection kit according to any one of (1) to (4) above, in which the antibody heavy-chain variable region polypeptide includes a polypeptide consisting of the amino acid sequence represented by SEQ ID No:1 and the antibody light-chain variable region polypeptide includes a polypeptide consisting of the amino acid sequence represented by SEQ ID No:2; and (6) the antigen-concentration measurement/detection kit according to any one of (1) to (4) above, in which the antibody heavy-chain variable region polypeptide includes a polypeptide consisting of the amino acid sequence represented by SEQ ID No:6 and the antibody light-chain variable region polypeptide includes a polypeptide consisting of the amino acid sequence represented by SEQ ID No:7.

Furthermore, the present invention relates to (7) an antigen-concentration measurement/detection method comprising the following steps (a) to (c) sequentially in this order: (a) bringing an antibody light-chain variable region polypeptide and an antibody heavy-chain variable region polypeptide labeled with a fluorescent dye or an antibody heavy-chain variable region polypeptide and an antibody light-chain variable region polypeptide labeled with a fluorescent dye into contact with (a1) an antigen in a test substance in a liquid phase, or (a2) an antigen in a non-human animal test subject administered with the antibody light-chain variable region polypeptide and the antibody heavy-chain variable region polypeptide labeled with a fluorescent dye or the antibody heavy-chain variable region polypeptide and the antibody light-chain variable region polypeptide labeled with a fluorescent dye, or (a3) an antigen in a test subject in vitro; (b) measuring the fluorescence intensity of the fluorescent dye in the case of (a1) and detecting fluorescence of the fluorescent dye in the cases of (a2) and (a3); and (c) computationally obtaining the level of the antigen contained in the test substance in the case of (a1) and visualizing the antigen contained in the test subject in the cases of (a2) and (a3), with reference to the positive correlation between the concentration of the antigen in the liquid phase and the fluorescence intensity of the fluorescent dye.

Furthermore, the present invention relates to (8) the antigen-concentration measurement/detection method according to (7) above, in which the antibody heavy-chain variable region polypeptide and the antibody light-chain variable region polypeptide are linked to form a single-chain antibody; (9) the antigen-concentration measurement/detection method according to (7) or (8) above, in which the fluorescent dye is a rhodamine-based fluorescent dye or an oxazine-based fluorescent dye; (10) the antigen-concentration measurement/detection method according to (9) above, in which the fluorescent dye is CR110, TAMRA or ATTO655; (11) the antigen-concentration measurement/detection method according any one of (7) to (10) above, in which the antibody heavy-chain variable region polypeptide includes a polypeptide consisting of the amino acid sequence represented by SEQ ID No:1, and the antibody light-chain variable region polypeptide includes a polypeptide consisting of the amino acid sequence represented by SEQ ID No:2; and (12) the antigen-concentration measurement/detection method according any one of (7) to (10) above, in which the antibody heavy-chain variable region polypeptide includes a polypeptide consisting of the amino acid sequence represented by SEQ ID No:6 and the antibody light-chain variable region polypeptide includes a polypeptide consisting of the amino acid sequence represented by SEQ ID No:7.

### BRIEF DESCRIPTION OF DRAWINGS

FIG. 1 This is a figure schematically showing CR110-labeled anti-BGP antibody light-chain variable region polypeptide (CR110-VL); TAMRA-labeled anti-BGP antibody heavy-chain variable region polypeptide (TAMRA-VH) and a complex thereof (CR110-VL/TAMRA-VH) of the present invention.

FIG. 2 CR110-VL and TAMRA-VH were allowed to react in the presence of BGP peptides in different concentrations and fluorescence spectra were measured at excitation light of 490 nm. The measurement results are shown in the figure.

FIG. 3 CR110-VL and TAMRA-VH were allowed to react in the presence of BGP peptides in different concentrations and fluorescence intensity at 525 nm (F525) and 575 nm (F575) was measured. The resultant change in fluorescence intensity is shown in the figure.

FIG. 4 CR110-VL and TAMRA-VH were allowed to react in the presence of BGP peptides in different concentrations. The fluorescence intensity at 525 nm (F525) and 575 nm (F575) was measured and a change in fluorescence intensity ratio (F575/F525) was analyzed. The results are shown in the figure.

FIG. 5 A CR110-labeled anti-BGP antibody light-chain variable region polypeptide and a TAMRA-labeled anti-BGP antibody heavy-chain variable region polypeptide of the present invention were allowed to react in the presence of BGP peptides in different concentrations. Analysis was made in accordance with fluorescence intensity multiple distribution analysis (FIDA) using a 488 nm laser and a 510 to 560 nm fluorescence filter. The results are shown in the figure. In the figure, CR110-VL represents the CR110-labeled anti-BGP antibody light-chain variable region polypeptide; TAMRA-VH represents the TAMRA-labeled anti-BGP antibody heavy-chain variable region polypeptide; and w.t.-VH represents a non-labeled anti-BGP antibody heavy-chain variable region polypeptide.

FIG. 6 A CR110-labeled anti-BGP antibody light-chain variable region polypeptide and a TAMRA-labeled anti-BGP antibody heavy-chain variable region polypeptide of the present invention were allowed to react in the presence of BGP peptides in different concentrations and analysis was made in accordance with the fluorescence intensity multiple distribution analysis (FIDA) using a 543 nm laser and a 560 to 620 nm fluorescence filter. The results are shown in the figure. In the figure, CR110-VL represents the CR110-labeled anti-BGP antibody light-chain variable region polypeptide; TAMRA-VH represents the TAMRA-labeled anti-BGP antibody heavy-chain variable region polypeptide; and w.t.-VL represents a non-labeled anti-BGP antibody light-chain variable region polypeptide.

FIG. 7 TAMRA-labeled anti-BGP antibody heavy-chain variable region polypeptide of the present invention was allowed to react with BGP peptides in different concentrations in the presence or absence of non-labeled anti-BGP antibody light-chain variable region polypeptide and fluorescence intensity was measured by use of 543 nm He—Ne laser. The results are shown in the figure. In the figure, +VL represents the results in the case where a reaction was performed in the presence of a non-labeled anti-BGP antibody light-chain variable region polypeptide; −VL represents the measurement results of fluorescence intensity in the case where a reaction was performed in the absence of non-labeled anti-BGP antibody light-chain variable region polypeptide.

FIG. 8 A TAMRA-labeled anti-BGP antibody heavy-chain variable region polypeptide of the present invention and BGP peptides in different concentrations were allowed to react in the presence or absence of a non-labeled anti-BGP antibody light-chain variable region polypeptide and the fluorescence-intensity ratio (+VL/−VL) was analyzed. The results are shown in the figure.

FIG. 9 Tryptophan residues present in an anti-BGP antibody heavy-chain variable region polypeptide of the present invention are shown in the figure. The figure shows the positions of tryptophan residues (W33, W36, W47, W106) in three-dimensional structure prediction models of a complex (VH/VL complex) of an anti-BGP antibody heavy-chain variable region polypeptide and an anti-BGP antibody light-chain variable region polypeptide, and a single anti-BGP antibody heavy-chain variable region polypeptide (VH). Note that the positions of these tryptophan residues respectively correspond to the amino acid positions 33, 36, 47, 103 in VH according to the Kabat numbering system.

FIG. 10 Either the wild-type (WT) or a mutant anti-BGP antibody heavy-chain variable region polypeptide (W33F, W36F, W47F, or W106F), and an anti-BGP antibody light-chain variable region polypeptide were allowed to react in the presence of BGP peptide in different concentration, and fluorescence intensity was measured by using a 543 nm He—Ne laser. The results are shown in the figure.

FIG. 11 Either the wild-type (WT) or a mutant anti-BGP antibody heavy-chain variable region polypeptide (W33F, W36F, W47F, or W106F) and an anti-BGP antibody light-chain variable region polypeptide were allowed to react in the presence of BGP peptides in different concentrations, and a change in diffusion time was analyzed by a Fluorescence Correlation Spectroscopy (FCS). The results are shown in the figure.

FIG. 12 This is a figure showing a three-dimensional structural prediction model of the complex (Fluorescent-labeled BGP-VH/VL) of a spacer-added fluorescent-labeled anti-BGP antibody heavy-chain variable region polypeptide (Fluorescent-labeled BGP-VH) and an anti-BGP antibody light-chain variable region polypeptide (BGP-VL).

FIG. 13 ATTO655-labeled anti-BGP antibody heavy-chain variable region polypeptide containing or not containing a spacer (GGGSGGGS; SEQ ID No:4) and an anti-BGP antibody light-chain variable region polypeptide were allowed to react in the presence of BGP peptides in different concentrations, and fluorescence intensity was measured. The results are shown in the figure.

FIG. 14 ATTO655-labeled anti-BGP antibody heavy-chain variable region polypeptide containing or not containing a spacer (GGGSGGGS; SEQ ID No:4) and an anti-BGP antibody light-chain variable region polypeptide were allowed to react in the presence of BGP peptides in different concentrations. The ratio of fluorescence intensity measured is shown in the figure.

FIG. 15 This is a figure showing a three-dimensional structural prediction model of a fluorescent-labeled single-chain antibody in which a fluorescent-labeled antibody heavy-chain variable region polypeptide and an antibody light-chain variable region polypeptide of the present invention are linked.

FIG. 16 This is a schematic figure of a primary structure of a fluorescent-labeled single-chain antibody in which a fluorescent-labeled antibody heavy-chain variable region polypeptide and an antibody light-chain variable region polypeptide of the present invention are linked.

FIG. 17 An ATTO655-labeled anti-BGP single-chain antibody containing or not containing a spacer and BGP peptides in different concentrations were allowed to react. The results of measured fluorescence intensity are shown in the figure.

FIG. 18 An ATTO655-labeled anti-BGP single-chain antibody containing or not containing a spacer and BGP peptides in different concentrations were allowed to react. The ratio of fluorescence intensity measured is shown in the figure.

FIG. 19 An ATTO655-labeled anti-BGP single-chain antibody of the present invention and BGP peptides in different concentrations were allowed to react, and fluorescence was detected by a fluorescence image analyzer (FMBIO-III; manufactured by Hitachi Software Engineering). The results are shown in the figure. In the figure, FL92 represents a spacer sequence consisting of the amino acid sequence represented by SEQ ID No:3; and 2TAG represents a spacer sequence consisting of MX (X represents a fluorescent-labeled amino acid).

FIG. 20 A fluorescent-labeled anti-BGP single-chain antibody containing or not containing a spacer, and BGP peptides in different concentrations were allowed to react, and fluorescence was quantitatively determined by the fluorescence image analyzer (FMBIO-III; manufactured by Hitachi Software Engineering). The results are shown in the figure. In the figure, FL92 represents a spacer sequence consisting of the amino acid sequence represented by SEQ ID No:3; and 2TAG represents a spacer sequence consisting of MX (X represents a fluorescent-labeled amino acid).

FIG. 21 A fluorescent-labeled anti-BGP single-chain antibody containing or not containing a spacer, and BGP peptides in different concentrations were allowed to react, and fluorescence was detected by the fluorescence image analyzer (FMBIO-III; manufactured by Hitachi Software Engineering). The results are shown in the figure. In the figure, FL92 represents a spacer sequence consisting of the amino acid sequence represented by SEQ ID No:3; and 2TAG represents a spacer sequence consisting of MX (X represents a fluorescent-labeled amino acid).

FIG. 22 A fluorescent-labeled anti-BGP single-chain antibody containing FL92 spacer (SEQ ID No:3), and BGP peptides in different concentrations were allowed to react. The ratio of fluorescence intensity measured by the fluorescence image analyzer (FMBIO-III; manufactured by Hitachi Software Engineering) and MF20/FluoroPoint-Light (manufactured by Olympus Corporation) is shown in the figure.

FIG. 23 A TAMRA-labeled anti-BGP single-chain antibody containing or not containing a spacer, and BGP peptides in different concentrations were allowed to react, and fluorescence was detected. The results are shown in the figure. G3S (1) indicates that G3S contains a spacer (linker) having a sequence represented by GGGS; G3S (2) indicates that G3S contains a spacer (linker) having a sequence represented by GGGSGGGS (SEQ ID No:4); and G3S (3) indicates that G3S contains a spacer (linker) having a sequence represented by GGGSGGGSGGGS (SEQ ID No:10).

FIG. 24 A TAMRA-labeled anti-BGP single-chain antibody protein and BGP in different concentrations were reacted in the presence of a PEST buffer solution and the human plasma of a final concentration of 50%, and fluorescence intensity was measured. The results are shown in the figure.

FIG. 25 An ATTO655-labeled anti-bisphenol A (BPA) antibody heavy-chain variable region polypeptide of the present invention and BPA in different concentrations were allowed to react in the presence or absence of a non-labeled anti-BPA antibody light-chain variable region polypeptide and fluorescence intensity was measured. The results are shown in the figure.

FIG. 26 An ATTO655-labeled anti-BPA antibody heavy-chain variable region polypeptide and BPA in different concentrations were allowed to react in the presence or absence of a non-labeled anti-BPA antibody light-chain variable region polypeptide, and fluorescence intensity was measured. The ratio of the fluorescence intensity measured is shown in the figure.

FIG. 27 A TAMRA-labeled anti-BPA single-chain antibody containing or not containing a spacer and BPA peptides in different concentrations were allowed to react, and fluorescence was detected. The results are shown in the figure. G3S (2) indicates that G3S contains a spacer (linker) having a sequence represented by GGGSGGGS (SEQ ID No:4); G3S (3) indicates that G3S contains a spacer (linker) having a sequence represented by GGGSGGGSGGGS (SEQ ID No:10); and G3S (5) indicates that G3S contains a spacer (linker) having a sequence represented by GGGSGGGSGGGSGGGSGGGS (SEQ ID No:11).

FIG. 28 A TAMRA-labeled anti-HEL single-chain antibody and HEL proteins in different concentrations were allowed to react and fluorescence was detected. The results are shown in the figure.

FIG. 29 A TAMRA-labeled anti-estradiol single-chain antibody and estradiol in different concentrations were allowed to react and fluorescence was detected. The results are shown in the figure.

FIG. 30 A TAMRA-labeled anti-SA single-chain antibody and BSA or HSA in different concentrations were allowed to react and fluorescence was detected. The results are shown in the figure.

FIG. 31 This is a figure showing that tryptophane at the position 36, 47 or 103 according to the Kabat numbering system is conserved in the amino acid sequence of a mouse antibody heavy-chain variable region. The figure shows a three-dimensional structural prediction model of an anti-BGP mouse antibody heavy-chain variable region and the possibility that W36, W47, W106 of the tryptophan residues contained in the anti-BGP mouse antibody heavy-chain variable region are particularly involved in the quenching.

FIG. 32 This is a figure showing that W33 is highly conserved in many types of mouse antibody heavy-chain variable regions.

FIG. 33 This is a figure showing that W36 is highly conserved in many types of mouse antibody heavy-chain variable regions.

FIG. 34 This is a figure showing that W47 is highly conserved in many types of mouse antibody heavy-chain variable regions.

FIG. 35 This is a figure showing that W106 is highly conserved in many types of mouse antibody heavy-chain variable regions. Note that Trp106 in the amino acid sequence of the above VH corresponds to the 103rd position according to the Kabat numbering system.

### MODE FOR CARRYING OUT THE INVENTION

An antigen-concentration measurement/detection kit of the present invention is a kit having an antibody light-chain variable region polypeptide and an antibody heavy-chain variable region polypeptide, either one of which is labeled with a fluorescent dye, wherein the kit enables a measurement of antigen concentration or visualization of an antigen with reference to the positive correlation between the concentration of the antigen in a liquid phase and the fluorescence intensity of the fluorescent dye.

More specifically, the antigen-concentration measurement/detection kit of the present invention is not particularly limited as long as it belongs to the following kits:

(1) an antigen concentration measurement kit having an antibody light-chain variable region polypeptide and an antibody heavy-chain variable region polypeptide labeled with a fluorescent dye, wherein the kit enables a measurement of the concentration of an antigen with reference to the positive correlation between the concentration of the antigen in a liquid phase and the fluorescence intensity of the fluorescent dye;

(2) an antigen detection kit having an antibody light-chain variable region polypeptide and an antibody heavy-chain variable region polypeptide labeled with a fluorescent dye, wherein the kit enables a visualization of an antigen with reference to the positive correlation between the level of the antigen in a test subject and the fluorescence intensity of the fluorescent dye;

(3) an antigen concentration measurement kit having an antibody heavy-chain variable region polypeptide and an antibody light-chain variable region polypeptide labeled with a fluorescent dye, wherein the kit enables a measurement of the concentration of an antigen with reference to the positive correlation between the concentration of the antigen in a liquid phase and the fluorescence intensity of the fluorescent dye; and

(4) a kit having an antibody heavy-chain variable region polypeptide and an antibody light-chain variable region polypeptide labeled with a fluorescent dye, wherein the kit enables a visualization of an antigen with reference to the positive correlation between the level of the antigen in a test subject and the fluorescence intensity of the fluorescent dye.

The antibody light-chain variable region polypeptide and the antibody heavy-chain variable region polypeptide may be prepared as two mutually independent polypeptide fragments as long as they can form a complex with a same antigen molecule interposed between them or may be prepared as a fused single-chain antibody with a linker, etc. interposed between them. Furthermore, the antigen is not particularly limited as long as it can be specifically recognized by the antibody heavy-chain variable region polypeptide and the antibody light-chain variable region polypeptide. Examples of the antigen include a protein, a peptide, a saccharide, a lipid, a glycolipid and a low molecular compound.

An antigen-concentration measurement/detection method of the present invention is an antigen-concentration measurement/detection method comprising the following steps (a) to (c) sequentially in this order: (a) bringing an antibody light-chain variable region polypeptide and an antibody heavy-chain variable region polypeptide labeled with a fluorescent dye or an antibody heavy-chain variable region polypeptide and an antibody light-chain variable region polypeptide labeled with a fluorescent dye into contact with (a1) an antigen in a test substance in a liquid phase, or (a2) an antigen in a non-human animal test subject administered with the antibody light-chain variable region polypeptide and the antibody heavy-chain variable region polypeptide labeled with a fluorescent dye or the antibody heavy-chain variable region polypeptide and the antibody light-chain variable region polypeptide labeled with a fluorescent dye, or in (a3) an antigen in a test subject in vitro; (b) measuring the fluorescence intensity of the fluorescent dye in the case of (a1) and detecting fluorescence of the fluorescent dye in the cases of (a2) and (a3); and (c) computationally obtaining the level of the antigen contained in the test substance in the case of (a1) and visualizing the antigen contained in the test subject in the cases of (a2) and (a3), with reference to the positive correlation between the concentration of the antigen in a liquid phase and the fluorescence intensity of the fluorescent dye.

More specifically, the antigen-concentration measurement/detection method of the present invention is not particularly limited as long as it belongs to the following methods:

an antigen-concentration measuring method (hereinafter sometimes referred to as “measurement method [I]”), comprising the following steps, sequentially in this order:

(a1-1) bringing an antibody light-chain variable region polypeptide and an antibody heavy-chain variable region polypeptide labeled with a fluorescent dye into contact with an antigen in a test substance, in a liquid phase,

(b) measuring the fluorescence intensity of the fluorescent dye, and

(c) computationally obtaining the level of the antigen contained in the test substance; that is, a step of computationally obtaining the level of the antigen contained in a test substance with reference to the positive correlation between the concentration of the antigen in a liquid phase and the fluorescence intensity of the fluorescent dye;

an antigen-concentration measuring method (hereinafter sometimes referred to as “measurement method [II]”) comprising the following steps, sequentially in this order:

(a1-2) bringing an antibody heavy-chain variable region polypeptide and an antibody light-chain variable region polypeptide labeled with a fluorescent dye into contact with an antigen in a test substance, in a liquid phase,

(b) measuring the fluorescence intensity of the fluorescent dye, and

(c) computationally obtaining the level of the antigen contained in the test substance; that is, a step of computationally obtaining the level of the antigen contained in a test substance with reference to the positive correlation between the concentration of the antigen in a liquid phase and the fluorescence intensity of the fluorescent dye;

an antigen detection method (hereinafter sometimes referred to as “non-human animal detection method [I]”) comprising the following steps, sequentially in this order:

(a2-1) bringing an antibody light-chain variable region polypeptide and an antibody heavy-chain variable region polypeptide labeled with a fluorescent dye into contact with an antigen in a non-human animal test subject administered with the antibody light-chain variable region polypeptide and the antibody heavy-chain variable region polypeptide labeled with a fluorescent dye,

(b) detecting the fluorescence of the fluorescent dye, and

(c) visualizing the antigen contained in the test subject, with reference to the positive correlation between the level of the antigen in the non-human animal test subject and the fluorescence intensity of the fluorescent dye;

an antigen detection method (hereinafter sometimes referred to as “non-human animal detection method [II]”) comprising the following steps, sequentially in this order:

(a2-2) bringing an antibody heavy-chain variable region polypeptide and an antibody light-chain variable region polypeptide labeled with a fluorescent dye into contact with an antigen in a non-human animal test subject administered with the antibody heavy-chain variable region polypeptide and the antibody light-chain variable region polypeptide labeled with a fluorescent dye,

(b) detecting the fluorescence of the fluorescent dye, and

(c) visualizing the antigen contained in the test subject, with reference to the positive correlation between the level of the antigen in the non-human animal test subject and the fluorescence intensity of the fluorescent dye;

an antigen detection method (hereinafter sometimes referred to as “in vitro detection method [I]”) comprising the following steps, sequentially in this order:

(a3-1) bringing an antibody light-chain variable region polypeptide and an antibody heavy-chain variable region polypeptide labeled with a fluorescent dye into contact with an antigen in a test subject in vitro;

(b) detecting the fluorescence of the fluorescent dye, and

(c) visualizing the antigen contained in the test subject, with reference to the positive correlation between the level of the antigen in the test subject and the fluorescence intensity of the fluorescent dye; and

an antigen detection method (hereinafter sometimes referred to as “in vitro detection method [II]”) comprising the following steps, sequentially in this order:

(a3-2) bringing an antibody heavy-chain variable region polypeptide and an antibody light-chain variable region polypeptide labeled with a fluorescent dye into contact with an antigen in a test subject in vitro;

(b) detecting the fluorescence of the fluorescent dye, and

(c) visualizing the antigen contained in the test subject, with reference to the positive correlation between the level of the antigen in the test subject and the fluorescence intensity of the fluorescent dye.

The antibody light-chain variable region polypeptide and the antibody heavy-chain variable region polypeptide may be prepared as mutually independent two polypeptide fragments as long as they can form a complex with a same antigen molecule interposed between them or may be prepared as a fused single-chain antibody with a linker, etc. interposed between them. Furthermore, the antigen is not particularly limited as long as it can be specifically recognized by the antibody heavy-chain variable region polypeptide and the antibody light-chain variable region polypeptide. Examples of the antigen include a protein, a peptide, a saccharide, a lipid, a glycolipid and a low molecular compound.

The antibody heavy-chain variable region polypeptide is not particularly limited as long as it contains an amino acid sequence specific to an antibody heavy-chain variable region encoded by exons of the V-region, D region and J region of an antibody heavy chain gene. An arbitral amino acid sequence may be further added to an N-terminal and/or C-terminal side of the amino acid sequence specific to the antibody heavy-chain variable region. Furthermore, as the amino acid sequence specific to the antibody heavy-chain variable region, an amino acid sequence having tryptophan as the amino acid positions 36, 47, 103 according to the Kabat numbering system is preferred. More specifically, the amino acid sequence represented by SEQ ID No:1 and the amino acid sequence represented by SEQ ID No:6 are preferably exemplified.

The antibody light-chain variable region polypeptide is not particularly limited as long as it contains an amino acid specific to an antibody light-chain variable region encoded by exons of V-region and J region of the antibody light-chain gene. An arbitral amino acid sequence may be further added to the N-terminal and/or C-terminal side of the amino acid sequence specific to the antibody light-chain variable region. Furthermore, as the amino acid sequence specific to the antibody light-chain variable region, an amino acid sequence having tryptophan as the 35th amino acid according to the Kabat numbering system is preferred. More specifically, the amino acid sequence represented by SEQ ID No:2 and the amino acid sequence represented by SEQ ID No:7 are preferably exemplified.

An antibody heavy-chain variable region polypeptide, an antibody light-chain variable region polypeptide and a single-chain antibody polypeptide containing both of an antibody heavy-chain variable region and an antibody light-chain variable region can be prepared by use of a known method such as a chemical synthesis method, a genetic recombination technique and a proteolytic decomposition method of an antibody molecule. Of them, the genetic recombination technique capable of preparing a polypeptide in a large amount and in a relatively easy operation is preferably employed for the preparation. When the polypeptide is prepared by the genetic recombination technique, DNA containing a nucleotide sequence encoding an amino acid sequence specific to an antibody light-chain variable region or an antibody light-chain variable region is introduced into a suitable vector to prepare an expression vector. By using an expression system of a host such as bacteria, yeast, an insect, an animal or plant cell and a cell-free translation system, a desired polypeptide can be expressed. When a polypeptide is expressed by the cell-free translation system, the polypeptide can be expressed in a reaction solution prepared by adding nucleotide triphosphates and various types of amino acids to a cell-free extract from e.g., Escherichia coli, wheat germ and rabbit reticulocyte.

The fluorescent dye is not particularly limited as long as it can be quenched in the state where it is labeled to an antibody heavy-chain variable region polypeptide or an antibody light-chain variable region polypeptide. Examples of the fluorescent dye include fluorescent dyes having rhodamine, coumarin, Cy, EvoBlue, oxazine, Carbopyronin, naphthalene, biphenyl, anthracene, phenanthrene, pyrene, carbazole, etc. as a basic skeleton and derivatives of these fluorescent dyes. Specific examples thereof include CR110: carboxyrhodamine 110: Rhodamine Green (trade name), TAMRA: carbocytetremethlrhodamine: TMR, ATTO655 (trade name), BODIPY FL (trade name): 4,4-difluoro-5,7-dimethyl-4-bora-3a, 4a-diaza-s-indancene-3-propionic acid, BODIPY 493/503 (trade name): 4,4-difluoro-1,3,5,7-tetramethyl-4-bora-3a,4a-diaza-s-indancene-8-propionicacid, BODIPY R6G (trade name): 4,4-difluoro-5-(4-phenyl-1,3-butadienyl)-4-bora-3a,4a-diaza-s-indancene-3-propionic acid, BODIPY 558/568 (trade name): 4,4-difluoro-5-(2-thienyl)-4-bora-3a,4a-diaza-s-indancene-3-propionic acid, BODIPY 564/570 (trade name): 4,4-difluoro-5-styryl-4-bora-3a,4a-diaza-s-indancene-3-propionic acid, BODIPY 576/589 (trade name): 4,4-difluoro-5-(2-pyrrolyl)-4-bora-3a,4a-diaza-s-indancene-3-propionic acid, BODIPY 581/591 (trade name): 4,4-difluoro-5-(4-phenyl-1,3-butadienyl)-4-bora-3a,4a-diaza-s-indancene-3-propionic acid, Cy3 (trade name), Cy3B (trade name), Cy3.5 (trade name), Cy5 (trade name), Cy5.5 (trade name), EvoBlue10 (trade name), EvoBlue30 (trade name), MR121, ATTO 390 (trade name), ATTO 425 (trade name), ATTO 465 (trade name), ATTO 488 (trade name), ATTO 495 (trade name), ATTO 520 (trade name), ATTO 532 (trade name), ATTO Rho6G (trade name), ATTO 550 (trade name), ATTO 565 (trade name), ATTO Rho3B (trade name), ATTO Rho11 (trade name), ATTO Rho12 (trade name), ATTO Thio12 (trade name), ATTO 610 (trade name), ATTO 611X (trade name), ATTO 620 (trade name), ATTO Rho14 (trade name), ATTO 633 (trade name), ATTO 647 (trade name), ATTO 647N (trade name), ATTO 655 (trade name), ATTO Oxa12 (trade name), ATTO 700 (trade name), ATTO 725 (trade name), ATTO 740 (trade name), Alexa Fluor 350 (trade name), Alexa Fluor 405 (trade name), Alexa Fluor 430 (trade name), Alexa Fluor 488 (trade name), Alexa Fluor 532 (trade name), Alexa Fluor 546 (trade name), Alexa Fluor 555 (trade name), Alexa Fluor 568 (trade name), Alexa Fluor 594 (trade name), Alexa Fluor 633 (trade name), Alexa Fluor 647 (trade name), Alexa Fluor 680 (trade name), Alexa Fluor 700 (trade name), Alexa Fluor 750 (trade name), Alexa Fluor 790 (trade name), Rhodamine Red-X (trade name), Texas Red-X (trade name), 5 (6)-TAMRA-X (trade name), STAMRA (trade name) and SFX (trade name). Of them, a rhodamine-based fluorescent dye such as CR110 and TAMRA, and an oxazine-based fluorescent dye such as ATTO655 can be particularly preferably mentioned.

A method of labeling an antibody light-chain variable region polypeptide and an antibody heavy-chain variable region polypeptide with a fluorescent dye is not particularly limited. For example, a method of directly labeling a polypeptide by use of a functional group at both sides or a side chain thereof or indirectly labeling a polypeptide via a crosslinking agent; and a method of site-specifically labeling while synthesizing a polypeptide by use of an in vitro transcription/translation system can be used. As the labeling method using an in vitro transcription/translation system, an amber suppression method (Ellman J et al. (1991) Methods Enzymol. 202: 301-36), a C-terminal labeling method (Japanese Unexamined Patent Application Publication No. 2000-139468) and an N-terminal labeling method (U.S. Pat. No. 5,643,722, Olejnik et al. (2005) Methods 36: 252-260) are known. In the amber suppression method, DNA or mRNA is prepared by substituting a codon encoding an amino acid of the target site to be labeled with one of the termination codons, i.e., an amber codon, and then a protein is synthesized based on the DNA or the mRNA using an in vitro transcription/translation system. At this time, if suppressor tRNA having a labeled non-natural amino acid bound thereto is added to a protein synthesis reaction solution, a protein having a labeled amino acid introduced in the amber codon substitution site can be synthesized. Furthermore, in the C-terminal labeling method, a protein having a label specifically introduced to the C-terminal can be synthesized by performing translation from DNA or mRNA to a protein in the in vitro transcription/translation system where a labeled puromycin is added in an optimal concentration.

The antigen-concentration measurement/detection kit of the present invention may contain reagents and tools usually employed in immune measurement kits of this type, such as a buffer solution, a tube or plate for measurement and an antigen serving as a reference substance. The antigen-concentration measurement/detection kit of the present invention can be suitably used in the antigen-concentration measurement/detection method of the present invention.

In the step (a1-1) of the measurement method [I] of the present invention, after an antibody light-chain variable region polypeptide and an antibody heavy-chain variable region polypeptide labeled with a fluorescent dye are added to a solution such as a buffer solution and a physiological saline solution, a test substance is added and incubation is performed to form a ternary complex consisting of the antibody light-chain variable region polypeptide/the antibody heavy-chain variable region polypeptide labeled with a fluorescent dye/an antigen specifically recognized by the antibody, in the solution. Furthermore, in the step (a1-2) of the measurement method [II], after an antibody heavy-chain variable region polypeptide and an antibody light-chain variable region polypeptide labeled with a fluorescent dye are added to a solution such as a buffer solution and a physiological saline solution, a test substance is added and incubation is performed to form a ternary complex consisting of the antibody heavy-chain variable region polypeptide/the antibody light-chain variable region polypeptide labeled with a fluorescent dye/an antigen specifically recognized by the antibody, in the solution. Examples of the test substance include substances that possibly contain the target antigen to be measured, such as body fluid including serum, plasma, saliva and urine, a culture supernatant, a cell extract, a bacterial-cell extract and industrial effluent. Furthermore, the incubation conditions are not particularly limited as long as they are generally used for an antibody/antigen reaction. The temperature condition is for example, 1 to 30° C. and preferably 18 to 25° C. The reaction time is, for example, 5 to 180 minutes and preferably 60 to 120 minutes. After completion of incubation, the solution can be subjected directly to the following step (b) without passing through a washing step, etc. This is a big feature of the antigen-concentration measurement/detection method of the present invention.

In the step (b) of the measurement method [I] and the measurement method [II] of the present invention, the fluorescence intensity of a fluorescent dye in the solution can be measured by applying excitation light to the solution prepared in the step (a1-1) or (a1-2). A fluorescence measurement apparatus to be used for measurement is not particularly limited. For example, MF20/FluoroPoint-Light (manufactured by Olympus Corporation) and FMBIO-III (manufactured by Hitachi Software Engineering) can be preferably mentioned. Furthermore, in the measurement, it is preferable to measure the following solutions prepared as a negative control of the solution prepared in the step (a1-1):

1) a solution not containing an antibody light-chain variable region polypeptide and containing an antibody heavy-chain variable region polypeptide labeled with a fluorescent dye alone,

2) a solution not containing an antibody light-chain variable region polypeptide and containing an antibody heavy-chain variable region polypeptide labeled with a fluorescent dye and a test substance alone,

3) a solution containing an antibody light-chain variable region polypeptide and an antibody heavy-chain variable region polypeptide labeled with a fluorescent dye and not containing a test substance, and

4) a solution containing an antibody light-chain variable region polypeptide, a non-labeled antibody heavy-chain variable region polypeptide and a test substance.

It is also preferable to measure the following solutions prepared as a negative control of the solution prepared in the step (a1-2):

1) a solution not containing an antibody heavy-chain variable region polypeptide and containing an antibody light-chain variable region polypeptide labeled with a fluorescent dye alone,

2) a solution not containing an antibody heavy-chain variable region polypeptide and containing an antibody light-chain variable region polypeptide labeled with a fluorescent dye and a test substance alone,

3) a solution containing an antibody heavy-chain variable region polypeptide and an antibody light-chain variable region polypeptide labeled with a fluorescent dye and not containing a test substance, and

4) a solution containing an antibody heavy-chain variable region polypeptide, a non-labeled antibody light-chain variable region polypeptide and a test substance.

In the step (c) of the measurement method [I] and the measurement method [II] of the present invention, the level of an antigen contained in a test substance can be calculated from the measurement value of fluorescence intensity obtained in the step (b). To describe it more specifically, since antigen concentration in the solution prepared in the step (a1-1) or (a1-2) and fluorescence intensity measured in the step (b) have a positive correlation, the level of an antigen contained in a test substance can be obtained by measuring fluorescence intensity of a test substance containing an antigen having a known concentration, preparing a reference curve showing the relationship between the antigen concentration and the fluorescence intensity, and computationally obtaining the antigen concentration corresponding to the fluorescence-intensity measurement value of a test substance containing the antigen having an unknown concentration. Furthermore, “computationally obtaining the level of the antigen” in the step (c) includes also the case of automatically obtaining the level of an antigen based on e.g., a conversion equation, previously determined based on the reference curve.

In the step (a2-1) of the non-human animal detection method [I] of the present invention, an antibody light-chain variable region polypeptide and an antibody heavy-chain variable region polypeptide labeled with a fluorescent dye are administered to a non-human animal test subject to form a ternary complex consisting of the antibody light-chain variable region polypeptide/antibody heavy-chain variable region polypeptide labeled with a fluorescent dye/antigen specifically recognized by the antibody, in the non-human animal test subject. Furthermore, in the step (a2-2) of the non-human animal detection method [II] of the present invention, an antibody heavy-chain variable region polypeptide and an antibody light-chain variable region polypeptide labeled with a fluorescent dye are administered systemically or locally to a non-human animal test subject to form a ternary complex consisting of the antibody heavy-chain variable region polypeptide/antibody light-chain variable region polypeptide labeled with a fluorescent dye/antigen specifically recognized by the antibody, in the non-human animal test subject. The non-human animal test subject is not particularly limited as long as it is an animal except a human. For example, a mouse, a rat, a hamster, a monkey and a pig are preferably mentioned. Furthermore, the “administration” method can be appropriately selected from parenteral local injection methods such as intramuscular injection, intraperitoneal injection, intravenous injection, hypodermic injection, embedding and coating, and peroral administration methods.

In the step (b) of the non-human animal detection method [I] and the non-human animal detection method [II] of the present invention, the fluorescence of the fluorescent dye in a non-human animal test subject injected with the antibody light-chain variable region polypeptide and the antibody heavy-chain variable region polypeptide, either one of which is labeled with a fluorescent dye, in the above step (a2-1) or (a2-2), is detected directly from the animal in a non-invasive manner. Alternatively, the fluorescence of the fluorescent dye is detected from the tissue or a cell taken from the non-human animal test subject. The above “detection” method is not particularly limited as long as the fluorescence of the fluorescent dye can be two-dimensionally or three-dimensionally detected by irradiating a body, tissue or cell of a non-human animal test subject with excitation light. Furthermore, at the time of detection, an image showing the structure of a body, tissue or cell of a non-human animal test subject is preferably obtained by use of e.g., an endoscope, X-ray, CT, MRI, ultrasonic wave and a microscope, simultaneously with the detection.

In the step (c) of the non-human animal detection method [I] and the non-human animal detection method [II] of the present invention, the antigen in a non-human animal test subject is visualized from the detection results of fluorescence of the fluorescent dye obtained in the step (b). More specifically, since level of an antigen in a non-human animal test subject and the fluorescence intensity of fluorescence detected in the step (b) have a positive correlation, localization (position) of the antigen can be found by comparing image data showing the structure of the body, tissue or cell of the non-human animal test subject with a two-dimensional or three-dimensional image of fluorescence detected in the step (b). For example, when an endoscope is used for detection, the tissue of a non-human animal test subject is irradiated with visible light to form an observable visible-light image; at the same time, an antibody light-chain variable region polypeptide and an antibody heavy-chain variable region polypeptide, either one of which is labeled, is applied to the tissue, thereby allowing them to be contact with the tissue, and thereafter, excitation light for labeled fluorescent dye is irradiated to the tissue to form a fluorescent image. Localization of the antigen in the tissue can be found by comparing the visible-light image to the fluorescent image.

In the step (a3-1) of the in vitro detection method [I] of the present invention, an antibody light-chain variable region polypeptide, an antibody heavy-chain variable region polypeptide labeled with a fluorescent dye and an antigen in a test subject are incubated in vitro to form a ternary complex consisting of the antibody light-chain variable region polypeptide/the antibody heavy-chain variable region polypeptide labeled with a fluorescent dye/the antigen specifically recognized by the antibody. Furthermore, in the step (a3-2) of the in vitro detection method [II], an antibody heavy-chain variable region polypeptide, an antibody light-chain variable region polypeptide labeled with a fluorescent dye and an antigen in a test subject are incubated in vitro to form a ternary complex consisting of the antibody heavy-chain variable region polypeptide/the antibody light-chain variable region polypeptide labeled with a fluorescent dye/the antigen specifically recognized by the antibody. As the test subject, a test subject that possibly contains the target antigen to be measured such as a cultured cell, a tissue slice and tissue or cell obtained from a living body can be mentioned. Other than these, e.g., a cell extract blotted on e.g., a nitrocellulose film and a PVDF film can be mentioned. Furthermore, the incubation conditions are not particularly limited as long as they can be generally used for an antibody/antigen reaction. The temperature condition is, for example, 1 to 30° C. and preferably 18 to 25° C. The reaction time is, for example, 5 to 180 minutes and preferably 60 to 120 minutes. After completion of incubation, the solution can be directly subjected to the following step (b) without subjecting to a washing step. This is a big feature of the antigen-concentration measurement/detection method of the present invention.

In the step (b) of the in vitro detection method [I] and the in vitro detection method [II], the fluorescence of the fluorescent dye in a test subject incubated with the antibody light-chain variable region polypeptide and the antibody heavy-chain variable region polypeptide, either one of which is labeled with a fluorescent dye in the above step (a3-1) or (a3-2), is two-dimensionally or three-dimensionally detected. As the “detection” method, e.g., a fluorescence microscope and a fluorescence image analyzer can be mentioned.

In the step (c) of the in vitro detection method [I] and the in vitro detection method [II], the antigen in a test subject can be visualized from the detection results of fluorescence of the fluorescent dye obtained in the step (b). More specifically, since level of an antigen in a test subject and the fluorescence intensity of fluorescence detected in the step (b) have a positive correlation, localization (position) of the antigen can be found based on a two-dimensional or three-dimensional image of fluorescence detected in the step (b).

In Examples shown below, the present invention will be more specifically described in detail. The following Examples are described in order to explain the present invention; however they should not construed as limiting the technical scope of the present invention.

### EXAMPLES

### Example 1

### 1. Establishment of Homogenous Fluorescence Based Immunoassay Using VH and VL Derived from Anti-BGP Antibody

### (Construction of Anti-BGP Antibody V-Region Gene Expression Vector)

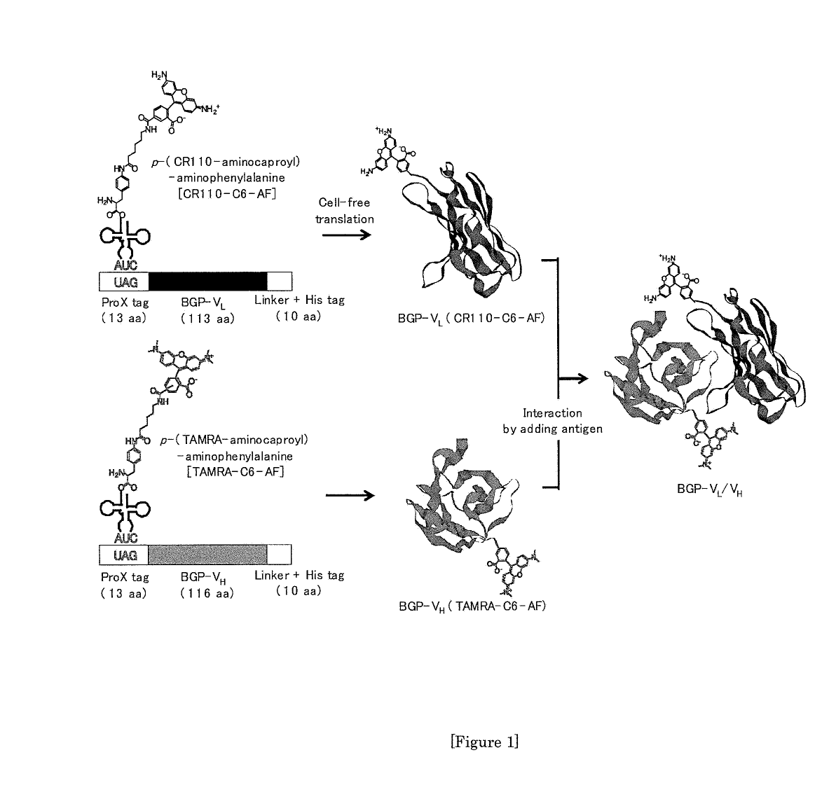

To a DNA sequence encoding a heavy-chain variable region (VH: SEQ ID No:1) or a light-chain variable region (VL: SEQ ID No:2) of an antibody against human osteocalcin (human Bone Gla Protein: BGP), a DNA sequence of ProX™tag (MSKQIEVNXSNET (X represents a fluorescent-labeled amino acid); SEQ ID No:3) containing an amber codon at the N-terminal was added. The resultant gene was inserted between the NcoI/HindIII sites of pIVEX2.3d vector (manufactured by Roche Diagnostics). The expression vector constructed is designed such that ProX™tag (MSKQIEVNXSNET (X represents a fluorescent-labeled amino acid): SEQ ID No:3) is added to the N-terminal of the inserted VH or VL, while His-tag is added to the C-terminal. FIG. 1 schematically shows that a CR110-labeled anti-BGP antibody light-chain variable region polypeptide (CR110-VL), a TAMRA-labeled anti-BGP antibody heavy-chain variable region polypeptide (TAMRA-VH) and the complex (CR110-VL/TAMRA-VH) thereof. In the same manner, the TAG codon was substituted with a TTT codon, a fluorescent-labeled amino acid residue was substituted with a phenylalanine residue to prepare the wild-type VH and VL expression vectors.

Furthermore, the following expression vectors were also prepared: mutant VH (W33F, W36F, W47F, or W106F) expression vectors, each of which was prepared by substituting one of four tryptophan codons (TGG; Trp33, Trp36, Trp47, Trp106) contained in the above VH gene with phenylalanine codons (TTT); a spacer-added VH expression vector, which was prepared by adding a spacer (GGGSGGGS; SEQ ID No:4) between ProXtag and the N-terminal of the VH gene; a single-chain antibody (scFv) expression vector, which was prepared by linking the VH gene and the VL gene via a linker (LVTVSSGGGGSGGGGSGGGGS; SEQ ID No:5, or GGGGSGGGGSGGGGS; SEQ ID No:9); a single-chain antibody (scFv) expression vector having a spacer (GGGS, GGGSGGGS; SEQ ID No:4 or GGGSGGGSGGGS; SEQ ID No:10) between the ProX™tag and the N-terminus of scFv; and an scFv expression vector having MX (X represents a fluorescent-labeled amino acid) at the N-terminus.

### (Preparation of Labeled Anti-BGP Antibody V-Region Protein)

Using RTS100 E. coli Disulfide Kit (manufactured by Roche Diagnostics), a fluorescent-labeled amino acid was introduced into the V-region protein N-terminal region by a cell-free translation system. A reaction solution (50 μL) was prepared by adding 7 μL of amino acid mix, 1 μL of methionine, 7 μL of Reaction mix, 25 μL of activated E. coli lysate, 5 μL of plasmid DNA (500 ng) and 5 μL of fluorescent-labeled aminoacyl-tRNAamber (0.8 nmol). As fluorescent-labeled aminoacyl-tRNA (TAMRA-X-AF-tRNAamber, CR110-X-AF-tRNAamber, and ATTO655-X-AF-tRNAamber) for preparing a fluorescent-labeled protein, CloverDirect™ tRNA Reagents for Site-Directed Protein Functionalization (manufactured by ProteinExpress) was used. The reaction solution was allowed to react at 20° C., 600 rpm for 2 hours and thereafter at 4° C. for 16 hours. After completion of the reaction, the reaction solution (1 μL) was subjected to SDS-PAGE (15%) and the expressed protein was observed by a fluorescence image analyzer (FMBIO-III; manufactured by Hitachi Software Engineering). Furthermore, Western blot was performed by use of an His-tag antibody to confirm the synthesis of target protein.

The V-region protein thus synthesized was purified by His-Spin Trap Column (manufactured by GE Healthcare). To the above reaction solution (50 μL), a Wash buffer (20 mM Phosphate buffer (pH 7.4)/0.5 M NaCl/60 mM imidazole/0.1% Polyoxyethylene (23) Lauryl Ether) was added up to a total volume of 400 This was applied to His-Spin Trap Column. Incubation was performed at room temperature for 15 minutes and washing was performed with Wash buffer three times. Subsequently, elution was performed twice with 200 μL of Elute buffer (20 mM Phosphate buffer (pH 7.4)/0.5 M NaCl/0.5 M imidazole/0.1% Polyoxyethylene (23) Lauryl Ether). Furthermore, the eluate, the buffer of which was exchanged with PBS (+0.05% Tween20), was concentrated by use of UltraFree-0.5 centrifugal devices (manufactured by Millipore). After purification, the concentration of the sample was measured by SDS-PAGE and FCS (MF20; manufactured by Olympus Corporation).

### Example 2

### Measurement of Fluorescence Spectra

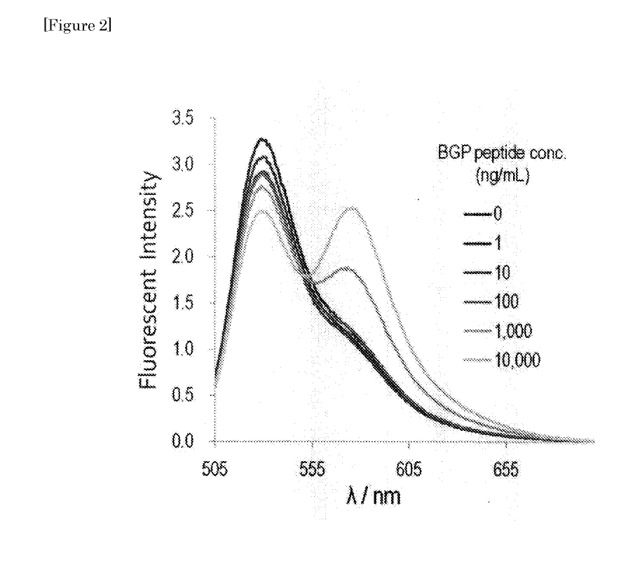

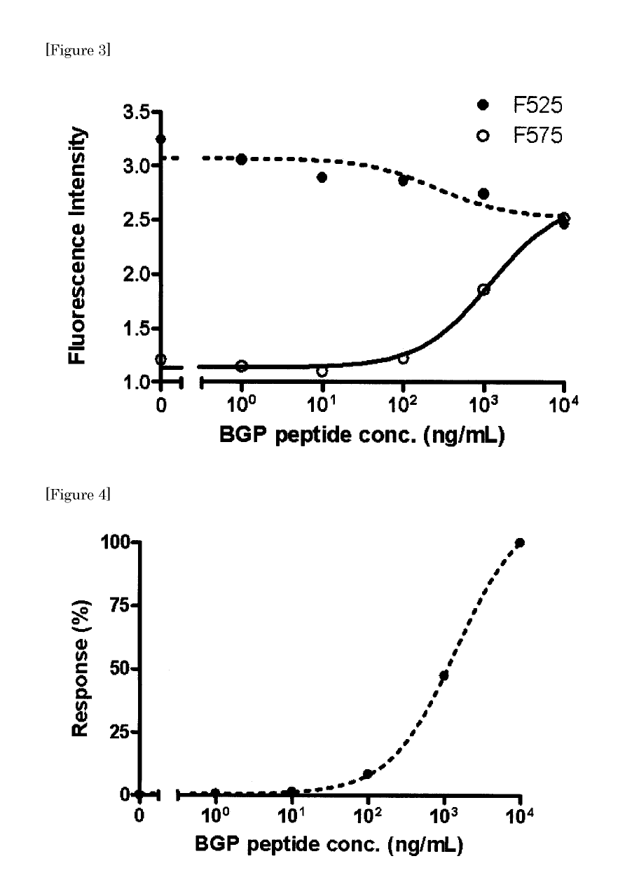

A sample was prepared by adding the TAMRA-labeled anti-BGP antibody VH protein and CR110-labeled anti-BGP antibody VL protein (1 μg/mL, 30 μL for each protein) prepared in Example 1 and 7-residue BGP C-terminal peptide (RRFYGPV; SEQ ID No:8) serving as an antigen and adjusted so as to have a total volume of 200 μL with PBS (+0.05% Tween20). After the mixture was allowed to stand still at 25° C. for 90 minutes, fluorescence spectra were measured by a fluorescence spectrophotometer (FluoroMax-4; manufactured by HORIBA Jobin Yvon GmbH). The excitation wavelength to the mixture of CR110-VL and TAMRA-VH was set to 490 nm, and the excitation wavelength to TAMRA-VH was set to 550 nm. With respect to the mixture of CR110-VL and TAMRA-VH, a fluorescence intensity ratio, I_{A}/I_{D}, was calculated, in which I_{A} and I_{D} were defined as the fluorescence intensity at 575 nm and 525 nm, respectively. The dissociation constant (Kd) value was calculated based on the fluorescence intensity ratio (I_{A}/I_{D}) or by the curve-fitting of the fluorescence intensity of a maximum fluorescent wavelength. At this time, as statistical analysis software, a sigmoidal dose-response model of Graphpad Prism (manufactured by Graphpad) was used. After CR110-VL and TAMRA-VH were allowed to react in the presence of BGP peptides in different concentrations, fluorescence spectra were measured by use of excitation light of 490 nm. The results are shown in FIG. 2. Changes of fluorescence intensity at 525 nm and 575 nm were measured, and the results are shown in FIG. 3. A change of the fluorescence intensity ratio of 525 nm and 575 nm (F575/F525) was analyzed and the results are shown in FIG. 4. A sample was prepared by adding a TAMRA-labeled anti-BGP antibody scFv protein (2 μg/mL, 25 μL) containing or not containing a spacer and a BGP peptide serving as an antigen, and adjusted so as to have a total volume of 200 μL with PBS (+0.05% Tween20, 0.2% BSA). Thereafter, the sample was allowed to stand still at 25° C. for 70 minutes, and fluorescence spectra were measured by use of a fluorescence spectrophotometer (FluoroMax-4; manufactured by HORIBA Jobin Yvon GmbH). The fluorescence intensity was calculated by curve-fitting. At this time, as statistical analysis software, a sigmoidal dose-response model of ImageJ software (http://rsbweb.nih.gov/ij/) was used. The measurement was performed at an excitation wavelength of 550 nm and a measurement wavelength of 580 nm.

### (Fluorescence Intensity Multiple Distribution Analysis)

A sample was prepared by adding the fluorescent-labeled VH protein and fluorescent-labeled VL protein (1 μg/mL, 7.5 μL for each protein) prepared in Example 1, or fluorescent-labeled scFv (1 μg/mL, 7.5 μL) and BGP peptide, and adjusted so as to have a volume of 50 μL with PBS (+0.05% Tween20). The resultant sample was added to a 384-well Glass Bottom Microplate (manufactured by Olympus Corporation) and incubated at 25° C. for 90 minutes. Measurement in accordance with the fluorescence intensity multiple distribution analysis (FIDA) was performed at 25° C. by use of MF20/FluoroPoint-Light (manufactured by Olympus Corporation). TAMRA and ATTO655 were excited by 543 nm- and 633 nm-laser, respectively. Data was obtained during 10 seconds per measurement and measurement was performed 10 times per sample. Based on these measurement values, an average value and a standard deviation were calculated.

### Example 3

### Evaluation of Binding Activity of TAMRA-VH and CR110-VL to BGP Peptide

First, an attempt to establish an antibody/antigen binding activity evaluation system using a fluorescent resonance energy transfer method (FRET) was made. In the FRET measurement, CR110 and TAMRA were used as a donor and an acceptor, respectively. Since fluorescence of the donor (CR110) is sufficiently overlapping with absorption spectrum of the acceptor (TAMRA), they can be used as an FRET pair. Provided that an orientation factor (κ²) is set to be ⅔, the Foerster distance (R₀) is calculated as 62 Å, which value is suitable for detecting the intermolecular interaction of proteins. When an antigen is not present, the interaction between VL and VH is poor and thus FRET from CR110 to TAMRA does not occur; whereas, when an antigen is present, VH and VL form a ternary complex with an antigen. As a result, it is predicted to induce FRET from CR110 to TAMRA.

For the purpose of determining whether FRET from CR110 to TAMRA is detected or not, by binding CR110-VL and TAMRA-VH with an antigen interposed between them, the following experiment was performed. CR110-VL and TAMRA-VH or non-labeled VH were incubated together with BGP antigen peptides in different concentrations (1 to 10,000 ng), and a change of fluorescence intensity of CR110 was analyzed in accordance with the fluorescence intensity multiple distribution analysis (FIDA). Measurement and detection were performed by using a 488 nm laser as excitation light and a 510 to 560 nm fluorescence filter. The results are shown in FIG. 5. When CR110-VL and TAMRA-VH were reacted, the fluorescence intensity of CR110 reduced in a BGP antigen peptide concentration-dependent manner. From the results, it was determined that FRET from CR110 to TAMRA was induced, as predicted, by forming a complex of CR110-VL and TAMRA-VH with an antigen peptide interposed between them. In contrast, unexpectedly, when CR110-VL was reacted with non-labeled VH, the fluorescence intensity of CR110 increased in a BGP antigen peptide concentration-dependent manner. From the results, the possibility that VL serves as a quencher to CR110; more specifically, following possibility was predicted. When CR110-VL is present alone, fluorescence of CR110 is quenched by VL; however, when the ternary complex of CR110-VL, VH and an antigen peptide is formed, the quenching effect is eliminated.

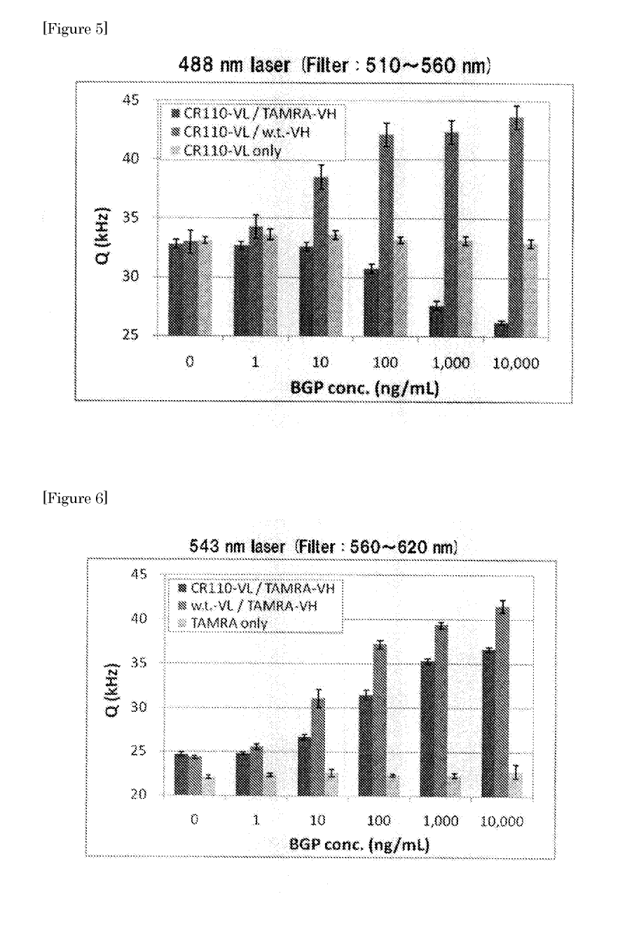

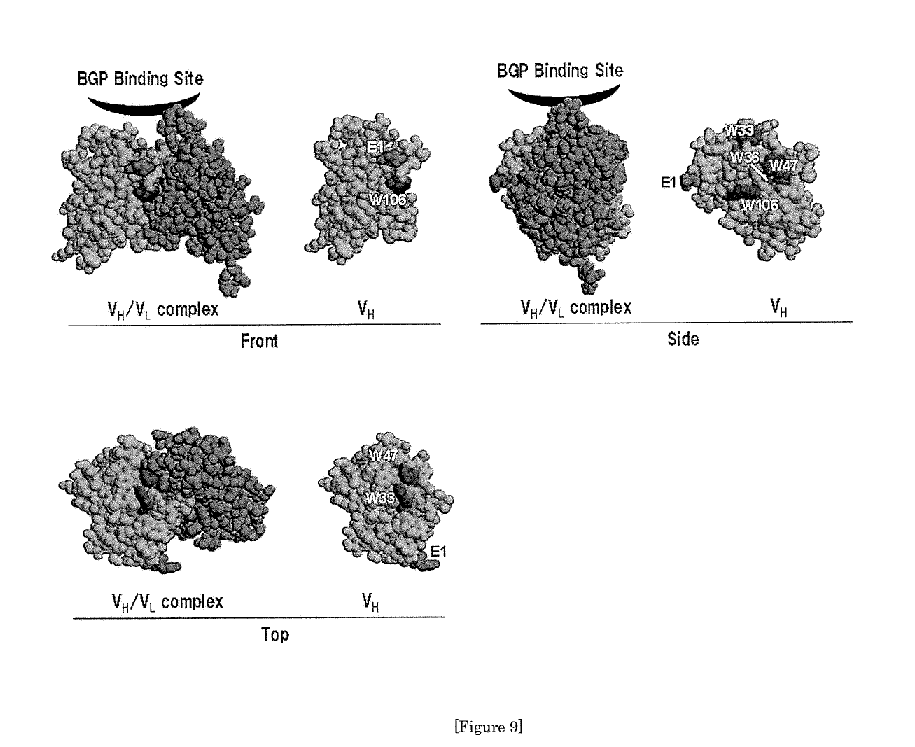

For the purpose of determining whether such a quenching effect by VL is observed also in VH, the following experiment was performed. TAMRA-VH and either CR110-VL or non-labeled VL were incubated together with BGP antigen peptides in different concentrations (1 to 10,000 ng), and the change of fluorescence intensity of TAMRA was analyzed in accordance with the fluorescence intensity multiple distribution analysis (FIDA). Measurement and detection were performed by using a 543 nm laser as excitation light and a 560 to 620 nm fluorescence filter. The results are shown in FIG. 6. When TAMRA-VH and CR110-VL were reacted, the fluorescence intensity of TAMRA increased in a BGP antigen peptide concentration-dependent manner. Similarly, when TAMRA-VH and non-labeled VL were reacted, the fluorescence intensity of TAMRA-VH also increased in a BGP antigen peptide concentration-dependent manner. From the results, similarly to the case of VL, the possibility that VH also serves as a quencher to TAMRA, more specifically, when TAMRA-VH is present alone, the fluorescence of TAMRA is quenched; however, when a ternary complex of TAMRA-VH, VL and an antigen peptide is formed, the quenching effect is eliminated, was predicted. From the results above, it was suggested that VH and VL have a quenching effect to a fluorescent dye, and furthermore the effect was eliminated upon formation of a ternary complex of VH/VL/antigen (FIG. 9).

### Example 4

### Establishment of Homogenous Fluorescence Based Immunoassay of Antigen Concentration Using Quenching Phenomenon

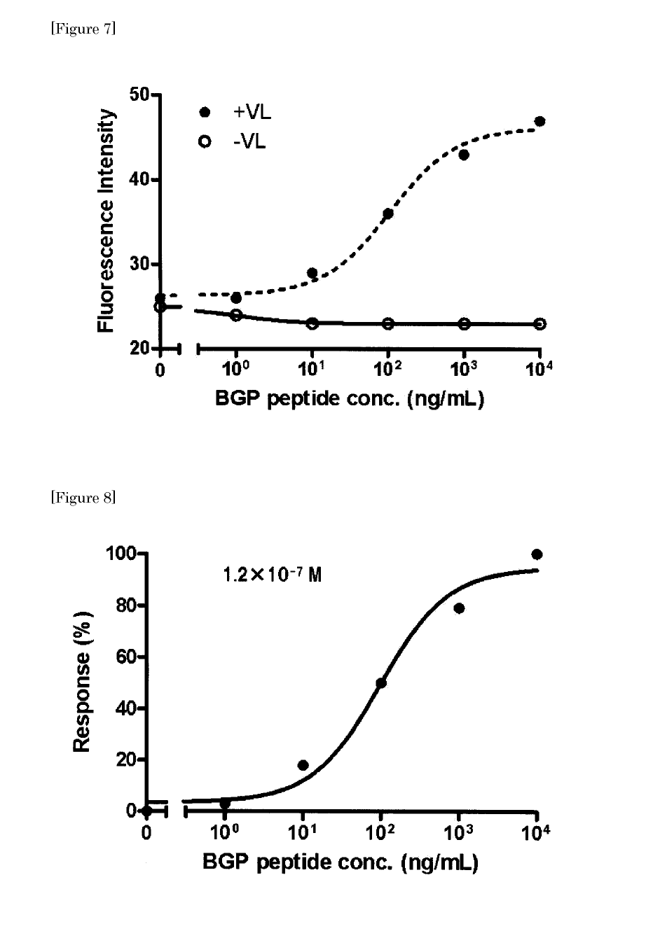

The inventors assumed that a novel immunoassay method may be established if a quenching phenomenon demonstrated in Example 3 is used. Based on the assumption, the following experiment was performed. TAMRA-VH and BGP peptides in different concentrations were reacted in the presence or absence of non-labeled VL, and fluorescence intensity was measured by use of an He—Ne laser of 543 nm. The results are shown in FIG. 7. In the presence of VL, the fluorescence intensity of TAMRA-VH increased in a BGP peptide concentration-dependent manner. In contrast, in the absence of VL, the fluorescence intensity of TAMRA-VH remained low in any concentration of BGP peptides used in the reaction. Furthermore, the fluorescence intensity ratio (+VL/−VL) of TAMRA-VH, in the presence of VL relative to TAMRA-VH in the absence of VL, was analyzed. As a result, the dissociation constant Kd was calculated as 1.2×10⁻⁷ [M] (FIG. 8). From the above results, it was demonstrated that a genuine novel homogenous fluorescence based immunoassay was successfully established using quenching phenomenon by VH and VL proteins.

### Example 5

### Study on Quenching Effect by Trp in VH

From the results of Example 3, it was demonstrated that the increase of TAMRA fluorescence intensity is higher than the decrease of CR110 fluorescence intensity, per titer of antigen. TAMRA is a rhodamine-based dye, and the studies so far made have reported that a rhodamine-based dye is quenched by an amino acid such as tryptophan (Trp). Hence, the inventors predicted that a Trp residue present in VH is involved in the quenching of TAMRA and postulated that when TAMRA-VH is present alone, fluorescence of TAMRA is quenched by the Trp residues present in the vicinity thereof; however, if TAMRA-VH forms a complex with VL and an antigen, the positional relationship between TAMRA and Trp may be changed to negate quenching. As shown in FIG. 9, VH has 4 Trp residues (Trp33, Trp36, Trp47, Trp106). From the analysis of predicted molecular model, it is estimated that Trp33, Trp36 and Trp106 are involved in hydrophobic interaction with VL; whereas Trp33 is involved in interaction with a BGP peptide. For the purpose for determining whether these Trp residues have an effect upon quenching, the following experiment was performed by using four types of mutant VHs, in which each Trp was substituted with Phe.

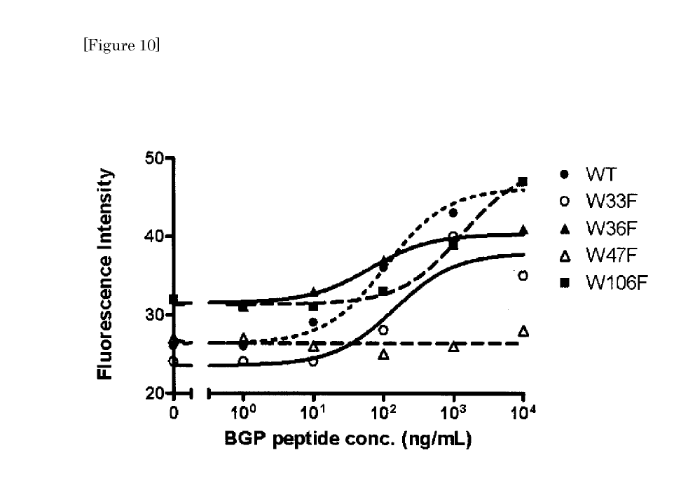

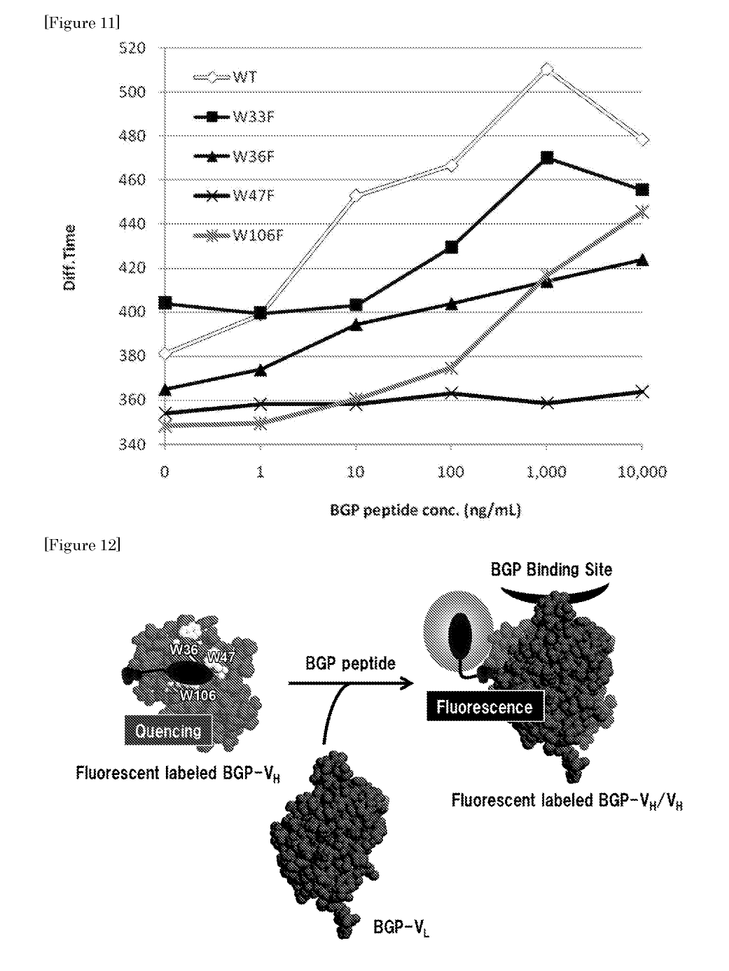

Either the wild-type or mutant anti-BGP antibody heavy-chain variable region polypeptide (W33F, W36F, W47F, or W106F) and VL were reacted in the presence of BGP peptides in different concentrations, and fluorescence intensity was measured by use of an He—Ne laser of 543 nm. The results are shown in FIG. 10 and Table 1. When the fluorescence intensity of a mutant fluorescent-labeled VH alone was measured, compared to the wild-type (WT), the fluorescence values of W106F and W36F exhibited an increase of 31% and 29%, respectively. The fluorescence of W47F exhibited an increase of 11%. In contrast, W33F exhibits a decrease of 9%. From these results, it was demonstrated that Trp36, Trp47 and Trp106 are mainly involved in the quenching of TAMRA fluorescence. Furthermore, in the cases of W33F, W36F, and W106F, if they are reacted with VL and a BGP peptide, fluorescence increases 1.5 times, 1.3 times and 1.5 times in an antigen-concentration dependent manner, respectively. The resultant fact that elimination of antigen-dependent quenching is attenuated by substitution of Trp by Phe suggests that Trp33, Trp36 and Trp106 are partly involved in quenching. In contrast, in the case of W47F, if it is reacted with a BGP peptide and VL, no increase of fluorescence was observed. Analysis results (FIG. 11) of diffusion time by FCS measurement show that binding activity of an antibody disappears by the mutation of Trp47. From this, it was demonstrated that Trp47 of VH is essential for binding between an antibody and an antigen.

From the above results, it was determined that Trp33 and Trp106 are critical Trp residues for quenching in view of two points: increasing fluorescence of a fluorescent-labeled VH alone and reducing an increase of fluorescence in an antigen-concentration dependent manner. Furthermore, involvement of Trp47 in quenching in an antigen-concentration dependent manner is not elucidated; however, it was elucidated that Trp47 is a very important site for formation of a VH and VL complex via an antigen. Note that Trp106 in the amino acid sequence of VH corresponds to the 103rd position according to the Kabat numbering system (Kabat, E. et al., “Sequences of proteins of immunological interest, 5th edn.,” U.S. Department of Health and Human Service, Public Service, National Institute of Health, Washington, D C, 1991).

TABLE 1
Relative Fluorescence Quenching of TAMRA-labeled VH proteins.

| | | | | Increased | Amount of | |
|---|---|---|---|---|---|---|
| | Fluorescence | Fluorescence | Fluorescence | amount of | change in | |
| V_{H} | intensity | intensity | intensity | fluorescence⁴ | fluorescence⁵ | |
| proteins | (I₀)*¹ | (I_{H})*² | (I_{HP})*³ | (I₀/I_{WT}) | (I_{HP}/I_{H}) | K_{d}*⁶ |
| WT | 24.7 | 25.6 | 47.1 | 1.00 | 1.84 | 1.2 × 10⁻⁷ |
| W33F | 21.8 | 23.7 | 34.8 | 0.88 | 1.47 | 1.6 × 10⁻⁷ |
| W36F | 31.8 | 31.5 | 41.4 | 1.29 | 1.31 | 7.2 × 10⁻⁸ |
| W47F | 26.7 | 27.4 | 27.6 | 1.08 | 1.01 | ND |
| W106F | 30.8 | 31.9 | 46.7 | 1.25 | 1.46 | 1.4 × 10⁻⁵ |

*¹Fluorescence intensity in the absence of (I₀) = VL or BGP pep. (fluorescence intensity of fluorescent labeled VH alone)
*²Fluorescence intensity in the presence of (I_{H}) = VL and in absence of BGP pep.
*³Fluorescence intensity in the presence of (I_{HP}) = VL and BGP pep. (concentration of BGP pep. herein is 10,000 ng/mL)
⁴Increased amount of fluorescence = rate of fluorescence intensity I₀ of each protein relative to fluorescence intensity I₀ of WT (represented by I_{wt})
⁵Amount of change in fluorescence = Amount of fluorescence increased in an antigen-concentration dependent manner
*⁶Dissociation constant

### Example 6

### Study on Quenching Effect by Addition of Oxazine-Based Fluorescent Dye and Spacer

Effect of a fluorescent dye and a spacer on sensitivity of homogenous fluorescence based immunoassay was studied. Efficiency of quenching greatly changes depending on the type of fluorescent dye. It has been reported that oxazine-based dyes are more effectively quenched than rhodamine-based dyes. Hence, the inventors prepared ATTO655-VH using an oxazine-based fluorescent dye ATTO655 as a label, and the same experiment as in Example 4 was performed. Furthermore, an ATTO655-VH (+spacer) was prepared by inserting GGGSGGGS (SEQ ID No:4) as a spacer between ATTO655 and VH, and the effect of the spacer on the quenching effect was studied. FIG. 12 shows a predicted three-dimensional structure model, a complex (Fluorescent-labeled BGP-VH/VL) of a spacer-added fluorescent-labeled anti-BGP antibody heavy-chain variable region polypeptide (Fluorescent-labeled BGP-VH) and an anti-BGP antibody light-chain variable region polypeptide (BGP-VL).

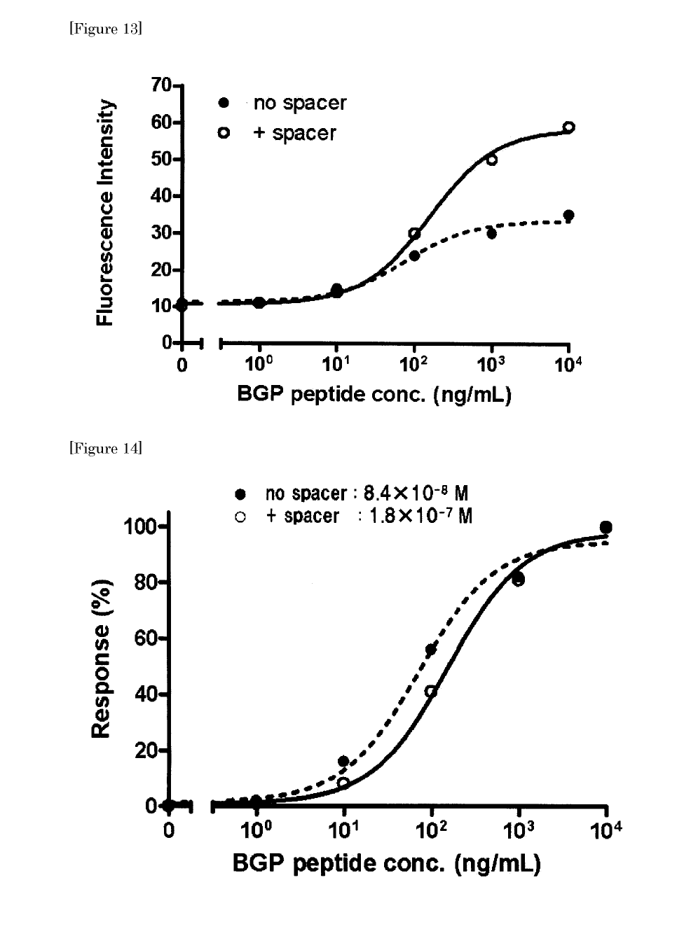

The same experiment as in Example 4 was performed by using ATTO655-VH with a spacer added thereto or without it. The results are shown in FIG. 13. In each case, the fluorescence intensity of ATTO655-VH increased in a BGP peptide concentration-dependent manner. Furthermore, the intensity of fluorescence obtained from ATTO655-VH having no spacer added thereto was three times as large as that of the case where TAMRA-VH was used. Furthermore, it was revealed that fluorescence intensity increases about twice by adding a spacer, compared to the case where no spacer is added. This is considered because the distance between quencher Trp and a fluorescent dye is reduced by adding a GGGS spacer, which has an effect on an increase of fluorescence intensity.

Furthermore, the fluorescence intensity ratio (+VL/−VL) of ATTO655-VH in the presence of VL to ATTO655-VH in the absence of VL, was analyzed, and the results are shown in FIG. 14. In the case of no spacer added, a dissociation constant Kd was calculated as 8.4×10⁻⁸ [M]; whereas in the case of a spacer added, the dissociation constant Kd was 1.8×10⁻⁷ [M]. Since it is considered that the sensitivity of the measurement system increases as the dissociation constant increases, the above results mean that a measurement system having further higher sensitivity was successfully established by providing a spacer between a fluorescent dye and VH.

### Example 7

### Establishment of Homogenous Fluorescence Based Immunoassay Using Single-Chain Antibody

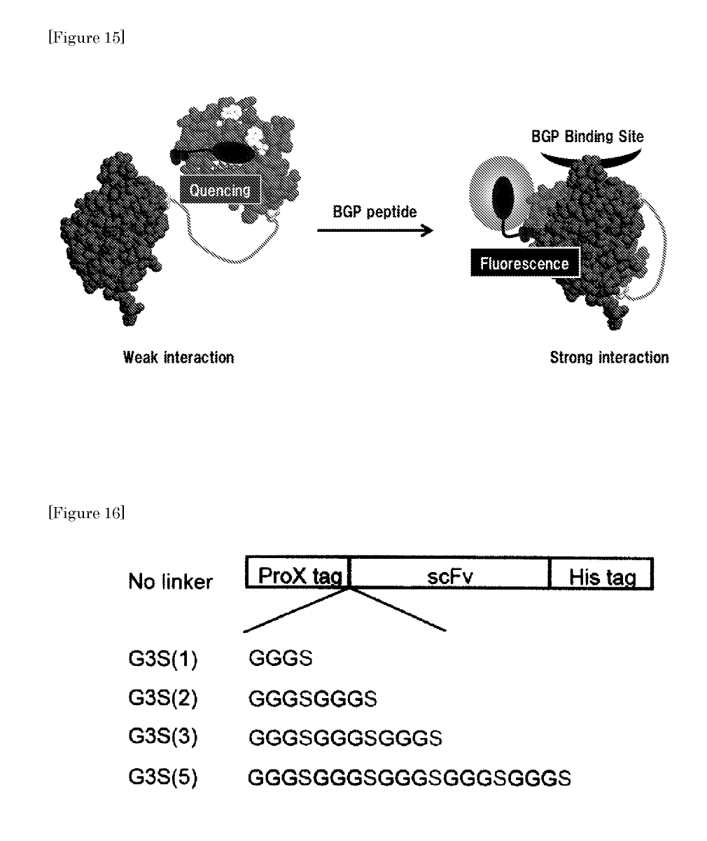

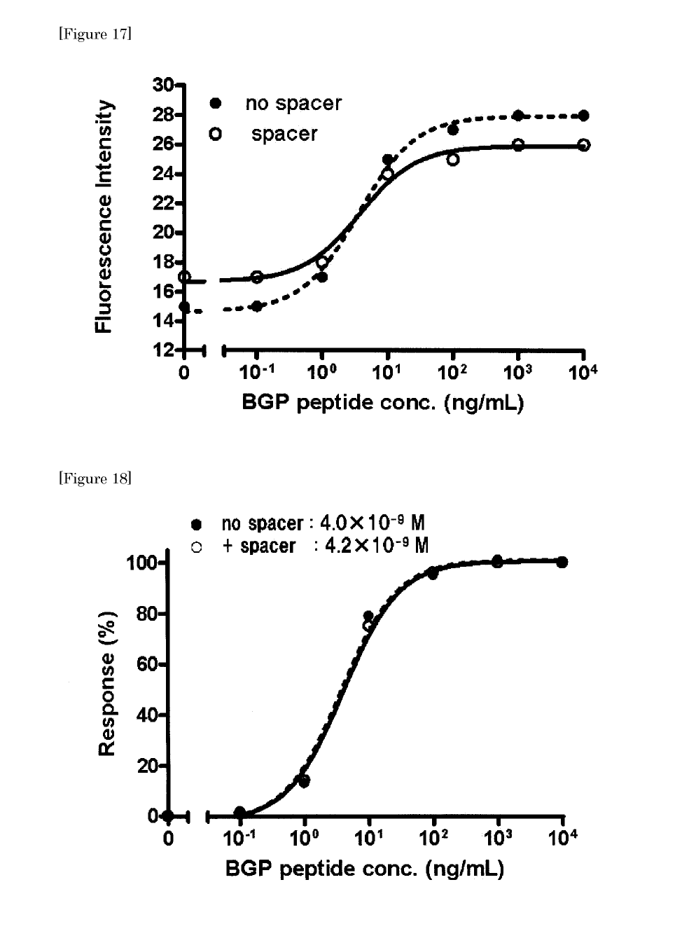

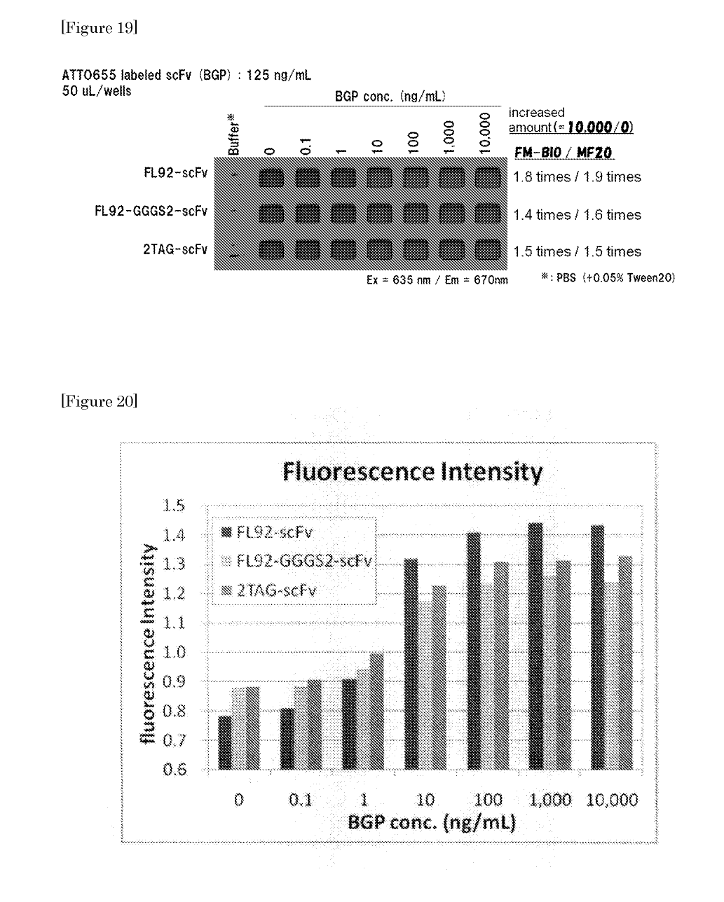

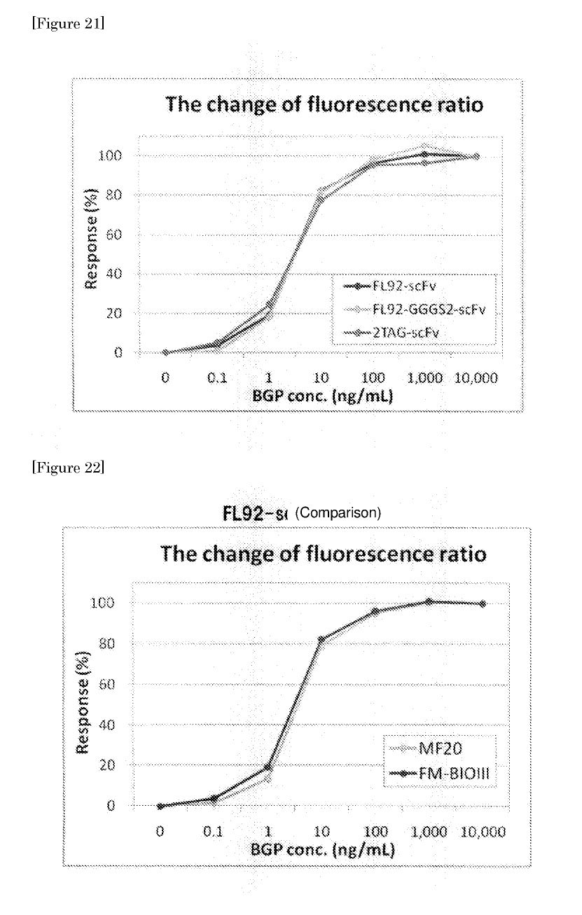

For the purpose of establishing a homogenous fluorescence based immunoassay using single-chain antibody (scFv) in which VH and VL were linked by a linker consisting of the amino acid sequence represented by SEQ ID No:5 or 9, the following experiment was performed. A predicted three-dimensional structure model of a fluorescent-labeled single-chain antibody formed by linking a fluorescent-labeled antibody heavy-chain variable region polypeptide and an antibody light-chain variable region polypeptide of the present invention is shown in FIG. 15, and the primary structure thereof is shown in FIG. 16. Similarly to the case where peptide fragments of VH and VL were used, also in the case of a fluorescent-labeled scFv, it was observed that fluorescence intensity increased in a BGP peptide concentration-dependent manner. The results of ATTO655-labeled anti-BGP antibody scFv linked by the linker represented by SEQ ID No:5 are shown in FIGS. 17 to 22; whereas, the results of TAMRA-labeled anti-BGP antibody scFv linked by the linker represented by SEQ ID No:9 are shown in FIG. 23.

### Example 8

### Fluorescence Immunoassay Method in the Human Plasma

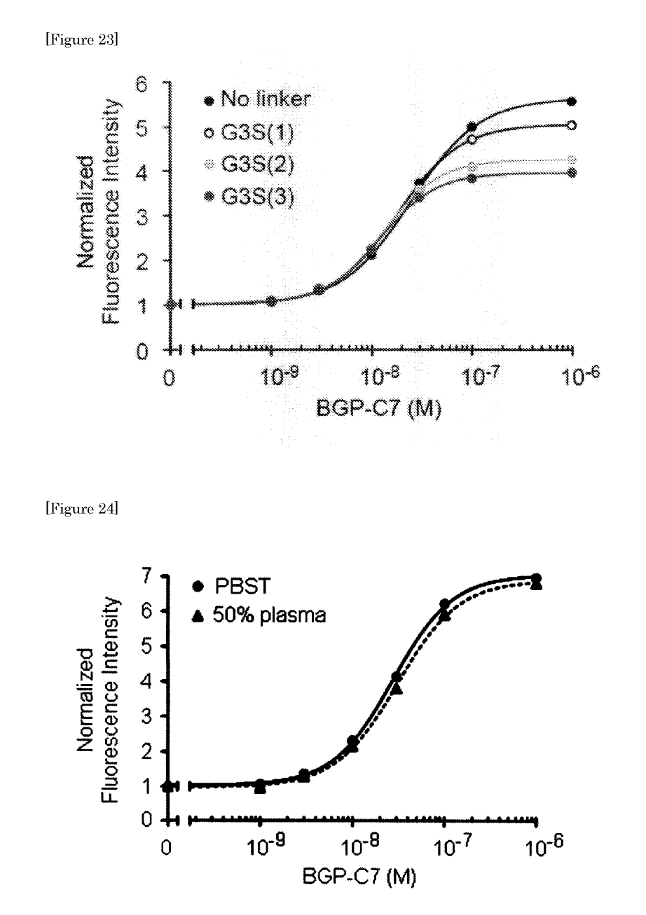

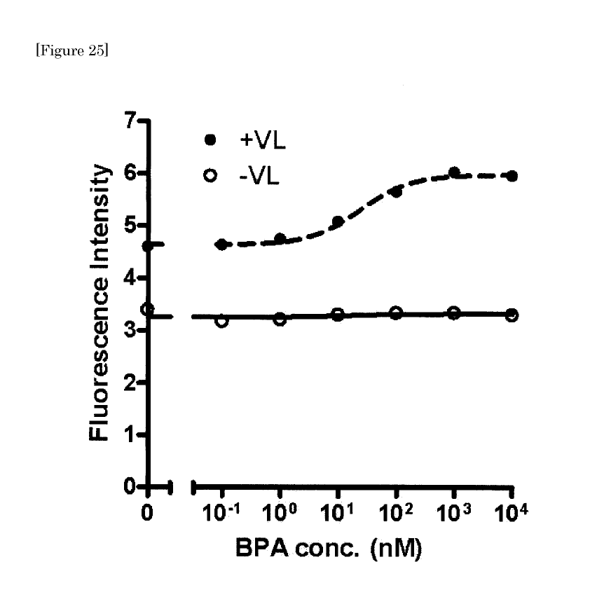

A sample was prepared by adding a TAMRA-labeled anti-BGP antibody scFv protein (2 μg/mL, 6.25 μL) and a BGP peptide serving as an antigen so as to contain 50% human plasma, and adjusted so as to have a total volume of 50 μl with PBS (+0.05% Tween20, 0.2% BSA). Thereafter, the sample was allowed to stand still at 25° C. for 90 minutes and then observed by a fluorescence image analyzer (FMBIO-III; manufactured by Hitachi Software Engineering). Measurement was performed at an excitation wavelength of 532 nm and at a measurement wavelength of 580 nm. The results are shown in FIG. 24. Even in the sample containing 50% human plasma, was observed that fluorescence intensity increased in a BGP peptide concentration-dependent manner.

### Example 9

### 2. Establishment of Homogenous Fluorescence Based Immunoassay Using VH and VL Derived from Anti-BPA Antibody

### (Construction of Anti-BPA Antibody Derived V-Region Gene Expression Vector)

To a gene encoding an anti-bisphenol A (BPA) antibody VH (SEQ ID No:6), a DNA sequence of ProX™tag (MSKQIEVNXSNET (X represents a fluorescent-labeled amino acid); SEQ ID No:3) containing an amber codon at the N-terminal was added. The resultant gene was inserted between the NcoI-HindIII sites of pIVEX2.3d vector (manufactured by Roche Diagnostics). The expression vector thus constructed is designed such that ProX™tag (MSKQIEVNXSNET (X represents a fluorescent-labeled amino acid); SEQ ID No:3) containing an amber codon is added to the N-terminal of the inserted VH and His-tag is added to the C-terminal. Furthermore, to a gene encoding an anti-BPA antibody VL (SEQ ID No:7), a DNA sequence in which amino acid X of the N-terminal ProX™tag is substituted with F was added in the same manner. The resultant gene was integrated in an NcoI-HindIII site of pIVEX2.3d vector (manufactured by Roche Diagnostics). The expression vector thus constructed is designed such that the sequence in which amino acid X of ProX™tag substituted with F is added to the N-terminal of the VL inserted and His-tag is added to the C-terminal. Moreover, a single-chain antibody (scFv) expression vector in a VH gene and a VL gene are bound by a linker (GGGGSGGGGSGGGGS; SEQ ID No:9) and three types of single-chain antibody (scFv) expression vectors having spacers: GGGSGGGS; SEQ ID No:4, GGGSGGGSGGGS; SEQ ID No:10 and GGGSGGGSGGGSGGGSGGGS; SEQ ID No:11, respectively, between ProX™tag and the N-terminal of scFv, were simultaneously prepared.

### (Preparation of Fluorescent-Labeled Protein in Cell-Free Translation System)

Using RTS100 E. coli Disulfide Kit (manufactured by Roche Diagnostics), a fluorescent-labeled amino acid was introduced into the N-terminal region of a V-region protein, by a cell-free translation system. A reaction solution (50 μL) was prepared by adding 7 μL of amino acid mix, 1 μL of methionine, 7 μL of Reaction mix, 25 μL of activated E. coli lysate, 5 μL of plasmid DNA (500 ng) and 5 μL of ATTO655-X-AF-tRNAamber (0.8 nmol). As ATTO655-X-AF-tRNAamber for preparing a fluorescent-labeled protein, CloverDirect™ tRNA Reagents for Site-Directed Protein Functionalization (manufactured by ProteinExpress) was used. The reaction solution was allowed to react at 20° C., 600 rpm for 2 hours and thereafter at 4° C. for 16 hours. After completion of the reaction, the reaction solution (1 μL) was subjected to SDS-PAGE (15%) and protein expression was observed by a fluorescence image analyzer (FMBIO-III; manufactured by Hitachi Software Engineering). Furthermore, Western blot was performed by use of an His-tag antibody to confirm the synthesis of a target protein.

The V-region protein thus synthesized was purified by His-Spin Trap Column (manufactured by GE Healthcare). To the above reaction solution (50 μL), a Wash buffer (20 mM Phosphate buffer (pH 7.4)/0.5 M NaCl/60 mM imidazole/0.1% Polyoxyethylene (23) Lauryl Ether) was added to bring the mixture to a total volume of 400 μL. This was applied to His-Spin Trap Column. Incubation was performed at room temperature for 15 minutes and washing was performed with Wash buffer three times. Subsequently, elution was performed with 200 μL of Elution buffer (20 mM Phosphate buffer (pH 7.4)/0.5 M NaCl/0.5 M imidazole/0.1% Polyoxyethylene (23) Lauryl Ether), twice. Furthermore, the eluate, the buffer of which was exchanged with PBS (+0.05% Tween20), was concentrated by use of UltraFree-0.5 centrifugal devices (manufactured by Millipore). The concentration of the sample was measured by SDS-PAGE and FCS (MF20; manufactured by Olympus Corporation).

### Example 10

### Measurement of Fluorescence Spectrum

A sample was prepared by adding the ATTO655-labeled anti-BPA antibody VH protein prepared in Example 9, a non-labeled anti-BPA antibody VL protein (1 μg/mL, 7.5 μL, respectively) and BPA serving as an antigen so as to bring a total volume of 50 μl of a 10% MeOH solution in PBS (+0.05% Tween20). After the mixture was allowed to stand still at 25° C. for 90 minutes, it was observed by a fluorescence image analyzer (FMBIO-III; manufactured by Hitachi Software Engineering). Measurement was performed at an excitation wavelength of 635 nm and at a measurement wavelength of 670 nm. A dissociation constant (Kd) value was calculated by the curve-fitting of a fluorescence measurement value. At this time, as statistical analysis software, a sigmoidal dose-response model of Graphpad Prism (manufactured by Graphpad) was used.

A sample was prepared by adding a TAMRA-labeled anti-BGP antibody scFv protein (2 μg/mL, 25 μL) containing or not containing a spacer and BGP serving as an antigen and adjusted so as to have a total volume of 200 μL with PBS (+0.05% Tween20, 0.2% BSA, 1% MeOH). Thereafter the sample was allowed to stand still at 25° C. for 10 minutes and then a fluorescence spectrum was measured by use of a fluorescence spectrophotometer (FluoroMax-4; manufactured by HORIBA Jobin Yvon GmbH). The fluorescence intensity was calculated by curve-fitting. At this time, as statistical analysis software, a sigmoidal dose-response model of ImageJ software (http://rsbweb.nih.gov/ij/), was used. The measurement was performed at an excitation wavelength of 550 nm and a measurement wavelength of 580 nm.

### Example 11

### Establishment of Homogenous Fluorescence Based Immunoassay Using Labeled V-Region Protein Derived from Anti-BPA Antibody

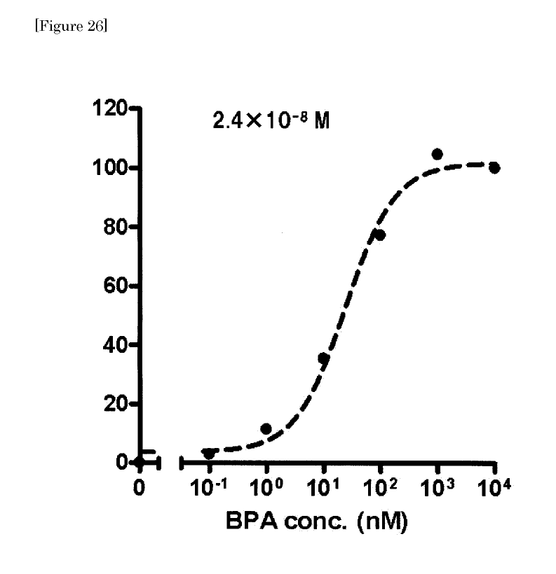

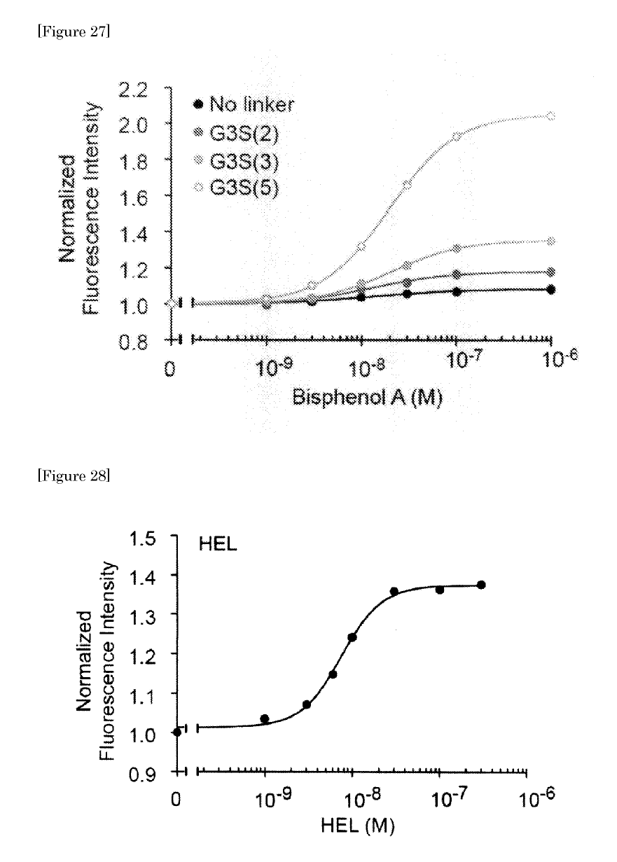

For the purpose of establishing a homogenous fluorescence based immunoassay using ATTO655-VH and VL without fluorescent-label, the following experiment was performed. ATTO655-VH and BPA in different concentrations were allowed to react in the presence or absence of non-labeled VL, and fluorescence intensity was measured (FIG. 25). In the presence of VL, the fluorescence intensity of ATTO655-VH increased in a BPA concentration dependent manner. In contrast, in the absence of VL, the fluorescence intensity of ATTO655-VH remained low in the case where any concentration of BPA was used in the reaction. Furthermore, the fluorescence intensity ratio of ATTO655-VH (+VL/−VL) in the presence of VL to ATTO655-VH in the absence of VL was analyzed. As a result, a dissociation constant (Kd) was calculated as 2.4×10⁻⁸ [M] (FIG. 26). Furthermore, for the purpose of establishing a fluorescence immunoassay method using a single-chain antibody (scFv) similarly to Example 7, the following experiment was performed. Also in the case where a TAMRA-labeled anti-BPA antibody scFv was used, it was observed that fluorescence intensity increased in a BPA concentration dependent manner (FIG. 27), similarly to the case where each of VH and VL peptide fragments was used.

### Example 12

### 3. Establishment of Homogenous Fluorescence-Based Immunoassay Using Single-Chain Antibodies Derived from Various Antibodies

### (Construction of Anti-HEL Antibody, Anti-Estradiol Antibody, Anti-SA Antibody-Derived V-Region Gene Expression Vector)

Expression vectors were constructed by inserting a DNA sequence, which has a ProX™tag (MSKQIEVNXSNET (X represents a fluorescent-labeled amino acid); SEQ ID No:3) containing an amber codon at the N-terminal of a DNA sequence of a single-chain antibody (scFv), an His-tag at the C-terminal, and a spacer (GGGSGGGS; SEQ ID No:4) between ProX™tag and the N-terminal of the scFv, between the NcoI and HindIII sites of pIVEX2.3d vector (manufactured by Roche Diagnostics). A DNA sequence of each single-chain antibody (scFv) is as follows:

an anti-chicken egg lysozyme (HEL) antibody scFv has a sequence formed by connecting a VH sequence (SEQ ID No:12) and a VL sequence (SEQ ID No:13) of an anti-HEL antibody in this order, by a linker sequence (GGGGSGGGGSGGGGS; SEQ ID No:9);

Estradiol (estradiol) antibody scFv has a sequence formed by connecting a VH sequence (SEQ ID No:14) and a VL sequence (SEQ ID No:15) of an anti-estradiol antibody in this order, by a linker sequence (GGGGSGGGGSGGGGS; SEQ ID No:9); and

SA (Serum Albumin) antibody scFv has a sequence formed by connecting a VH sequence (SEQ ID No:16) and a VL sequence (SEQ ID No:17) of an anti-SA antibody in this order, by a linker sequence (GGGGSGGGGSGGGGS; SEQ ID No:9).

### (Preparation of Fluorescent-Labeled Anti-HEL Antibody, Anti-Estradiol Antibody, Anti-SA Antibody V-Region Protein)

Using RTS100 E. coli Disulfide Kit (manufactured by Roche Diagnostics), a fluorescent-labeled amino acid was introduced to the V-region protein N-terminal region by a cell-free translation system. A reaction solution (50 μL) was prepared by adding 7 μL of amino acid mix, 1 μL of methionine, 7 μL of reaction mix, 25 μL of activated E. coli lysate, 5 μL of plasmid DNA (500 ng) and 5 μL of fluorescent-labeled aminoacyl-tRNAamber (0.8 nmol). As the fluorescent-labeled aminoacyl-tRNA (TAMRA-X-AF-tRNAamber) for preparing a fluorescent labeled protein, CloverDirect™ tRNA Reagents for Site-Directed Protein Functionalization (manufactured by ProteinExpress) was used. The reaction solution was allowed to react at 20° C., 600 rpm for 2 hours and thereafter at 4° C. for 16 hours. After completion of the reaction, the reaction solution (1 μL) was subjected to SDS-PAGE (15%), and protein expression was observed by a fluorescence image analyzer (FMBIO-III; manufactured by Hitachi Software Engineering). Furthermore, Western blot was performed by use of an His-tag antibody to confirm the synthesis of a target protein.

The V-region protein thus synthesized was purified by His-Spin Trap Column (manufactured by GE Healthcare). To the above reaction solution (50 μL), a Wash buffer (20 mM Phosphate buffer (pH 7.4)/0.5 M NaCl/60 mM imidazole/0.1% Polyoxyethylene (23) Lauryl Ether) was added to bring a total volume to 400 μL. This was applied to His-Spin Trap Column. Incubation was performed at room temperature for 15 minutes and washing was performed with Wash buffer three times. Subsequently, elution was performed with 200 μL of Elution buffer (20 mM Phosphate buffer (pH 7.4)/0.5 M NaCl/0.5 M imidazole/0.1% Polyoxyethylene (23) Lauryl Ether), twice. Furthermore, the eluate, the buffer of which was exchanged with PBS (+0.05% Tween20), was concentrated by use of UltraFree-0.5 centrifugal devices (manufactured by Millipore). After purification, the concentration of the sample was measured by SDS-PAGE and FCS (MF20; manufactured by Olympus Corporation).

### Example 13

### Measurement of Fluorescence Spectrum

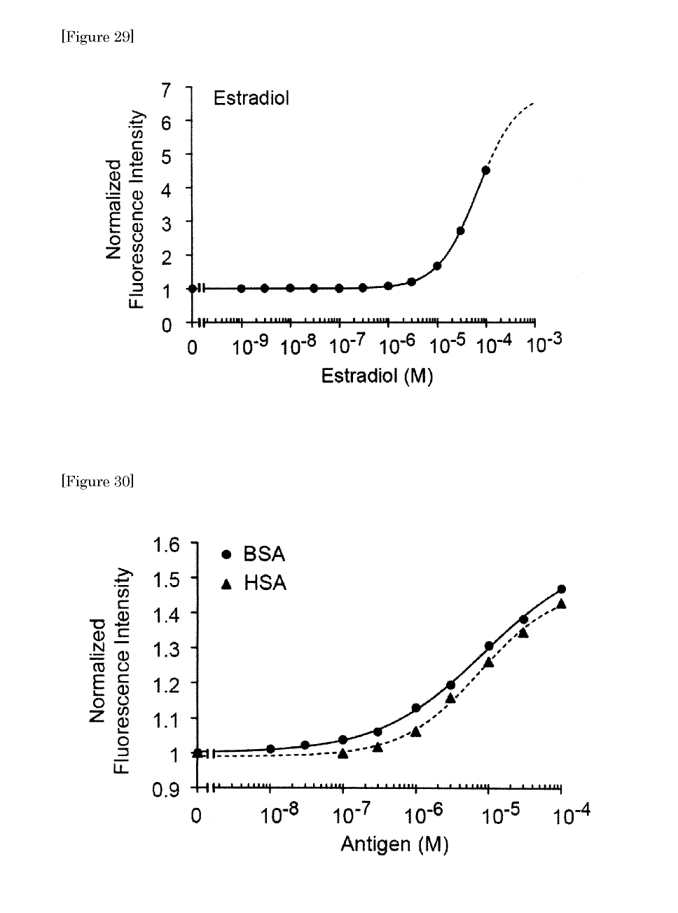

A sample was prepared by adding a TAMRA-labeled anti-HEL antibody scFv protein (2 μg/mL, 25 μL) and an HEL protein serving as an antigen and adjusted so as to have a total volume of 200 μL with PBS (+0.05% Tween20, 1% BSA). A sample was prepared by adding a TAMRA-labeled anti-estradiol antibody scFv protein (2 μg/mL, 25 μL) and estradiol serving as an antigen and adjusted so as to have a total volume of 200 μL with PBS (+0.05% Tween20, 1% BSA). A sample was prepared by adding a TAMRA-labeled anti-SA antibody scFv protein (2 μg/mL, 25 μL) and BSA (bovine serum albumin) or HSA (human serum albumin) serving as an antigen and adjusted so as to have a total volume of 200 μL with PBS (+0.05% Tween20, 0.2% gelatin). After the samples were allowed to stand still at 25° C. for 5 minutes, fluorescence spectra were measured by a fluorescence spectrophotometer (FluoroMax-4; manufactured by HORIBA Jobin Yvon GmbH) and the fluorescence intensity was calculated by curve fitting. At this time, as statistical analysis software, a sigmoidal dose-response model of ImageJ software (http://rsbweb.nih.gov/ij/), was used. The measurement was performed at an excitation wavelength of 550 nm and a measurement wavelength of 580 nm. As a result, it was observed that fluorescence intensity increased in an antigen-concentration dependent manner (FIGS. 28 to 30). As described above, it was demonstrated that fluorescence immunoassay method can be performed by using various types of fluorescent-labeled single-chain antibodies (scFv).

### Example 14

### 4. Conservation of Trp Residue in Mouse Antibody VH

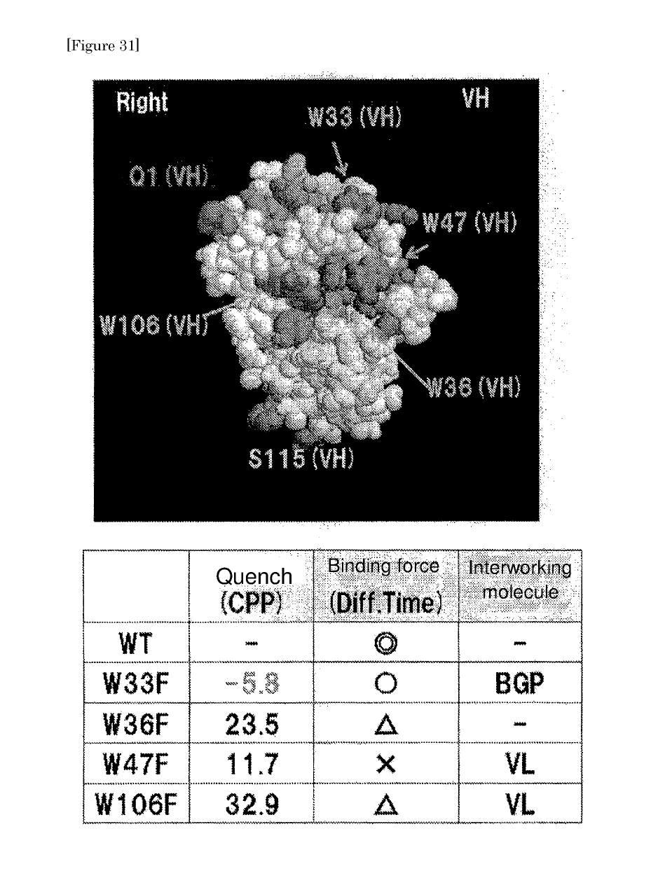

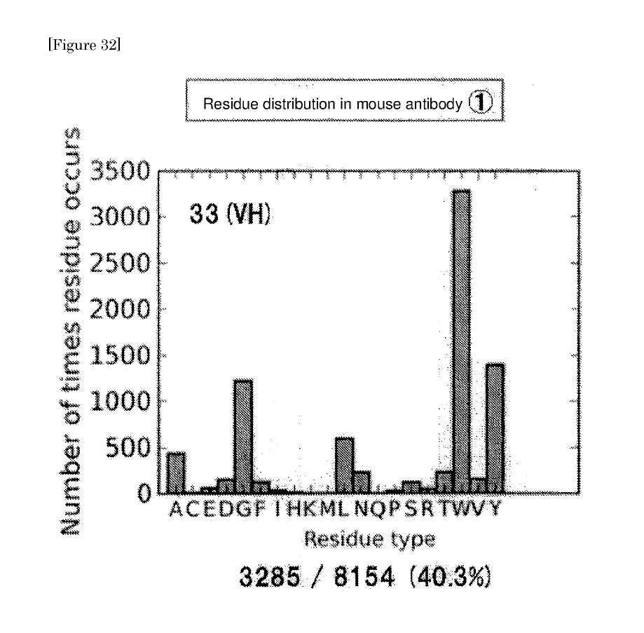

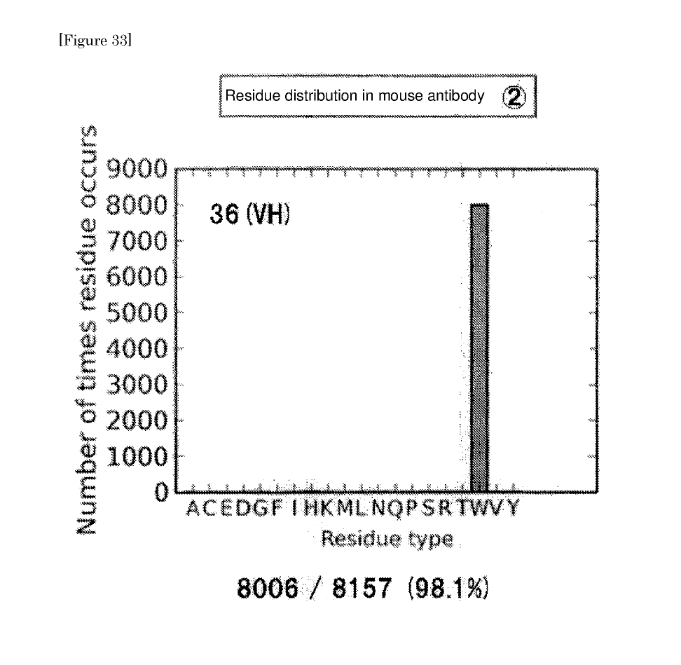

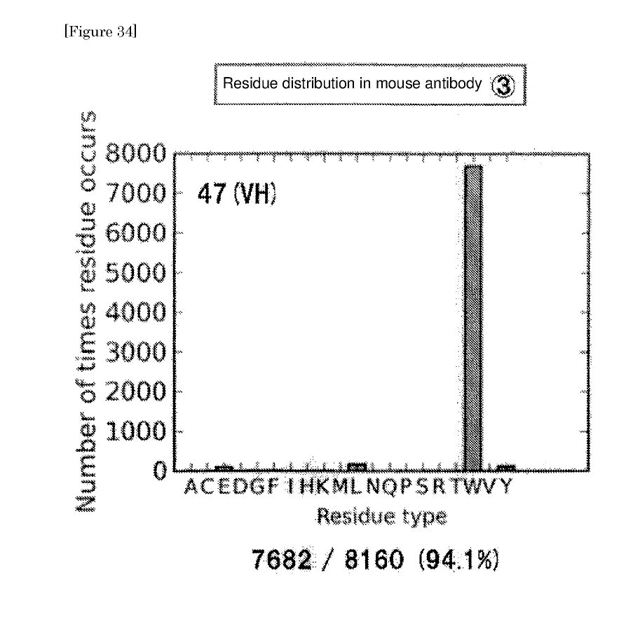

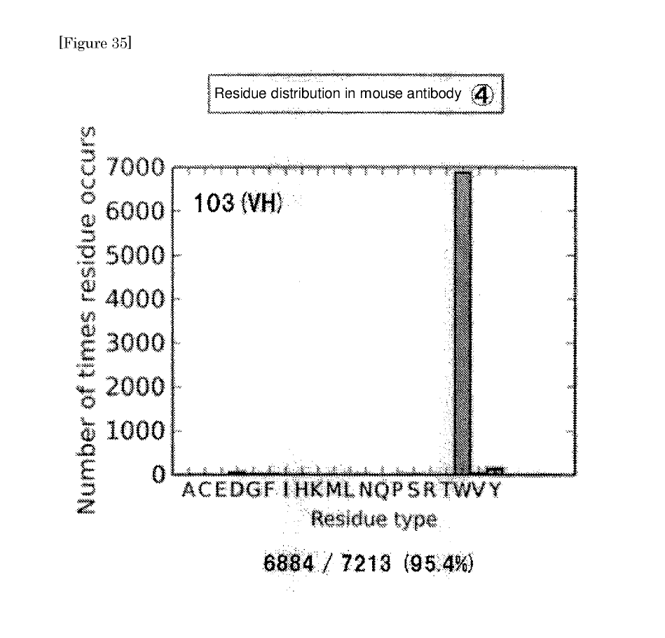

As shown in Example 4, FIG. 9 and Table 1, it was found that the 33rd, 36th and 106th Trp (according to the Kabat numbering system) of the VH amino acid sequence have an important role in quenching a fluorescent-dye labeled to an anti-BGP antibody. It was further found that the 47th Trp is essential for binding between an antibody (VH and VL) to an antigen (Note that Trp106 in the VH amino acid sequence corresponds to the 103rd position according to the Kabat numbering system). Hence, the present inventors investigated whether these tryptophan residues are conserved in mouse antibody VH-regions other than the anti-BGP antibody. Amino acid residue distribution of mouse antibodies was analyzed by use of Abysis database (Dr. Andrew C. R. Martin's Group; http://www.bioinf.org.uk/abs/reference.html). Furthermore, residue numbers of each antibody residue according to the Kabat sequence notational system were investigated based on AbCheck (Dr. Andrew C. R. Martin's Group; Martin, A.C.R. Accessing the Kabat Antibody Sequence Database by Computer PROTEINS: Structure, Function and Genetics, 25 (1996), 130-133; http://www.bioinf.org.uk/abs/seqtest.html). As is apparent from the results shown in FIGS. 31 to 35, the conservation rates of four Trp residues in the mouse antibody VH-region were as follows: Trp33: 40%; Trp36: 98%; Trp47: 94%; and Trp33: 95%. These results show that the four Trp residues of VH, which are critical for carrying out a homogenous fluorescence based immunoassay, are conserved in many mouse-antibody VH sequences.

The positions of tryptophan residues contained in VH (SEQ ID No:1) and VL (SEQ ID No:2) of an anti-BGP antibody and VH (SEQ ID No:6) and VL (SEQ ID No:7) of anti-BPA antibody used in aforementioned Examples were numbered in accordance with the Kabat sequence notational system. The results are shown in Table 2. Furthermore, the positions of tryptophan residues contained in anti-BGP antibody scFv, anti-BPA antibody scFv, anti-HEL antibody scFv, anti-SA antibody scFv and anti-estradiol antibody scFv were numbered in accordance with the Kabat sequence notational system. The results are shown in Table 3.

| | | | | | |
|---|---|---|---|---|---|
| V_{H} | Kabat sequence | Trp33 | Trp36 | Trp47 | Trp103 |
| | notational system | | | | |
| | BGP | Trp33 | Trp36 | Trp47 | Trp106 |
| | BPA | Trp35 | Trp37 | Trp48 | Trp109 |
| V_{L} | Kabat sequence | Trp35 | | | |
| | notational system | | | | |
| | BGP | Trp40 | | | |
| | BPA | Trp39 | | | |

Residue number*

| | CDRH1 | | | | | CDRH3 | | | | | CDRL3 | |
|---|---|---|---|---|---|---|---|---|---|---|---|---|
| | H33 | H34 | H35 | H36 | H47 | H95 | H103 | L35 | L47 | L91 | L92 | L94 |
| αBGP | W | I | H | W | W | S | W | W | L | T | T | V |
| αBisphenolA | Y | Y | W | W | W | V | W | W | I | S | W | I |
| αHEL | Y | W | S | W | Y | W | W | W | L | S | N | W |
| αBSA | A | M | S | W | W | S | W | W | L | A | D | S |
| αEstradiol | T | I | H | W | W | Y | W | W | W | W | S | Y |

### INDUSTRIAL APPLICABILITY

According to the present invention, it is possible to provide a homogenous fluorescence based immunoassay using an antibody (fragment) labeled with a fluorescent dye with reference to elimination of quenching of a fluorescent dye. In the homogenous fluorescence based immunoassay of the present invention, the concentration of a target substance can be measured by directly monitoring the fluorescence intensity of a mixture solution having an antibody and a test substance mixed therein without immobilization and washing of an antibody or an antigen. Thus, it is presumed that a low-molecular compound can be quickly and easily detected. Furthermore, since Trp residues of an antibody VH-region having an effect on quenching are conserved in many types of antibodies, the homogenous fluorescence based immunoassay of the present invention can be used in measurement of various antigen concentrations.

1-6. (canceled)

7. An antigen-concentration measurement/detection method comprising the following steps (a) to (c) sequentially in this order:
(a) bringing an antibody light-chain variable region polypeptide and an antibody heavy-chain variable region polypeptide labeled with a fluorescent dye or an antibody heavy-chain variable region polypeptide and an antibody light-chain variable region polypeptide labeled with a fluorescent dye into contact with
(a1) an antigen in a test substance in a liquid phase, or
(a2) an antigen in a non-human animal test subject administered with the antibody light-chain variable region polypeptide and the antibody heavy-chain variable region polypeptide labeled with a fluorescent dye or the antibody heavy-chain variable region polypeptide and the antibody light-chain variable region polypeptide labeled with a fluorescent dye, or
(a3) an antigen in a test subject in vitro;
(b) measuring the fluorescence intensity of the fluorescent dye in the case of (a1) and detecting fluorescence of the fluorescent dye in the cases of (a2) and (a3); and
(c) computationally obtaining level of the antigen contained in the test substance in the case of (a1) and visualizing the antigen contained in the test subject in the cases of (a2) and (a3), with reference to the positive correlation between the concentration of the antigen in the liquid phase and the fluorescence intensity of the fluorescent dye.

8. The antigen-concentration measurement/detection method according to claim 7, wherein the antibody heavy-chain variable region polypeptide and the antibody light-chain variable region polypeptide are linked to form a single-chain antibody.

9. The antigen-concentration measurement/detection method according to claim 7, wherein the fluorescent dye is a rhodamine-based fluorescent dye or an oxazine-based fluorescent dye.

10. The antigen-concentration measurement/detection method according to claim 9, wherein the fluorescent dye is carboxyrhodamine 110, carboxytetramethylrhodamine or 11-(3-carboxypropyl)-1-ethyl-10,10-dimethyl-8-(sulfonatomethyl)-2,3,4,8,9,10-hexahydro-1H-13-oxa-1,6,11-triazapentacen-11-ium.

11. The antigen-concentration measurement/detection method according to claim 7, wherein the antibody heavy-chain variable region polypeptide includes a polypeptide consisting of the amino acid sequence represented by SEQ ID No:1 and the antibody light-chain variable region polypeptide includes a polypeptide consisting of the amino acid sequence represented by SEQ ID No:2.

12. The antigen-concentration measurement/detection method according to claim 7, wherein the antibody heavy-chain variable region polypeptide includes a polypeptide consisting of the amino acid sequence represented by SEQ ID No:6 and the antibody light-chain variable region polypeptide includes a polypeptide consisting of the amino acid sequence represented by SEQ ID No:7.
